(19) United States
(12) Patent Application Publication
(10) Pub. No.: US 2023/0233650 A1
(43) Pub. Date: Jul. 27, 2023

(54) NOVEL FATTY ACID MODIFIED UROCORTIN 2 DERIVATIVES AND THE USES THEREOF

(71) Applicant: Novo Nordisk A/S, Bagsvaerd (DK)

(72) Inventors: Chien Li, Seattle, WA (US); Nicholas Raymond Cox, Brainbridge Island, WA (US); Jason O'Neil, Lake Forest Park, WA (US); Patrick William Garibay, Holte (DK); Berit Christoffersen Gyrstinge, Ringsted (DK); Hans Dennis Aasberg, Frederikssund (DK); Lennart Lykke, Koebenhavn V (DK); Nikolaj Kulahin Roed, Birkeroed (DK)

(21) Appl. No.: 17/865,438

(22) Filed: Jul. 15, 2022

Related U.S. Application Data
(63) Continuation of application No. PCT/EP2022/069248, filed on Jul. 11, 2022.
(60) Provisional application No. 63/220,788, filed on Jul. 12, 2021.

(30) Foreign Application Priority Data
Oct. 5, 2021 (EP) 21201010.2

(51) Int. Cl. A61K 38/22; A61P 3/10; A61K 47/54

(52) U.S. Cl. CPC A61K 38/2228; A61K 47/542; A61P 3/10; A61K 38/00

(57) ABSTRACT

Ucn2 derivatives comprising a peptide and a substituent with high potency, high physical and high chemical stability, suitable for administration to humans, and their medical use in treatment and/or prevention of obesity and/or diabetes.

### CROSS-REFERENCE TO RELATED APPLICATIONS

This application is a continuation of International Application PCT/EP2022/069248, filed Jul. 11, 2022, which claims priority to European Patent Application 21201010.2, filed Oct. 5, 2021; this application claims priority under 35 U.S.C. § 119 to U.S. Provisional Application 63/220,788, filed Jul. 12, 2021; the contents of which are incorporated by reference in their entirety.

### TECHNICAL FIELD OF THE INVENTION

The present invention relates to stable derivatives of the human peptide Urocortin 2 that are able to activate the Corticotropin-Releasing Factor receptor 2 (CRF₂ receptor), as well as uses thereof.

### INCORPORATION-BY-REFERENCE OF THE SEQUENCE LISTING

The instant application contains a Sequence Listing which has been submitted in XML format via the USPTO patent electronic filing system and is hereby incorporated by reference in its entirety. Said XML file, created on Jul. 12, 2022, is named 210058US02.xml and is 11 kilobytes in size.

### BACKGROUND

Urocortin 2 (Ucn2) is a 38 amino acid peptide found in mammals including rodents and humans. It is one of the three known endogenous mammalian urocortins (Ucn1-3) and is belonging to the corticotropin-releasing factor (CRF) family, also known as corticotropin-releasing hormone family, that, besides Ucn2, contains corticotropin-releasing hormone (CRH), Ucn1 and Ucn3 in mammals. The CRF family is vitally important for coordinating physiological and behavioural responses to stress.

The function of CRH and the Ucn’s are mediated through two G-protein coupled receptors, namely the type 1 and the type 2 CRF receptors (herein referred to as CRF₁ receptor and CRF₂ receptor, respectively), which have distinct pharmacology and agonist affinity. The CRF₁ receptor binds CRH and Ucn1 with similarly high affinity but does not bind Ucn2 or Ucn3. On the contrary, the CRF₂ receptor binds all Ucn’s with higher binding affinity than for CRH.

The two CRF receptors mediate different aspects of the stress response. Specifically, the CRF₁ receptor is responsible for activating the hypothalamic-pituitary-adrenal axis to stimulate cortisol secretion from the adrenal gland and for mediating stress associated behaviour. The CRF₂ receptor on the other hand, plays a critical role in coordinating energy homeostasis in stress response including cessation of appetite, mobilisation of fuel storage and enhancement of energy expenditure (Bale and Vale, 2004, Annu. Rev. Pharmacol. Toxicol. 44:525-57).

The CRF₂ receptor is expressed in discrete areas of the brain including the hypothalamus and central activation in rodents suppresses food intake and may also increase energy expenditure (Chen et al., 2012, Front. Endocrinol., 3(180):1-12; Stengel and Tache, 2014, Front. Neurosci., 8:52). In the periphery, the CRF₂ receptor is mainly expressed in skeletal muscle, heart, blood vessels, pancreatic β-cells, and the stomach. Stimulation of the muscle CRF₂ receptors has been shown to enhance insulin-stimulated glucose uptake, promote thermogenesis and increase muscle mass (Borg et al., 2019, Diabetes, 68:1403-1414; Solinas et al., 2006, Endocrinology 147(1):31-38, Hinkle et al., 2004, Journal of Muscle Research and Cell Motility 25:539-547), whereas stimulation of the pancreatic CRF₂ receptors modulates glucagon and insulin secretion (Li et al., 2003, Endocrinology, 144(7):3216-3224). Furthermore, activation of the CRF₂ receptor in the cardiovascular system results in vasodilation and positive chronotropic, inotropic and lusitropic effects in the heart, leading to an increased cardiac output. In addition, CRF₂ receptor activation has been shown to confer cardio-protection in ischemia (Venkatasubramanian et al., 2010, Biochem Pharmacol, 80:289-296).

The native Ucn2 peptide is short acting, with a half-life in circulation of ~10 min in humans (Davis et al., 2007, J Am Coll Cardiol., 49(4):461-471). Thus, Ucn2 displays suboptimal pharmacokinetic properties with respect to obtaining a suitable daily exposure.

Based on the demonstrated cardiometabolic effects in e.g. animal models and humans (only cardiovascular effects in humans), CRF₂ receptor-selective Ucn2 analogues are expected to have a positive effect on body weight, body composition, muscle mass and -function, glucose metabolism and cardiac function in humans and are thus suggested to be useful for the treatment of e.g. obesity, sarcopenic obesity, sarcopenia, diabetes and heart failure.

Therefore, there is an increased interest in developing Ucn2-based compounds that can be used for treatment of diseases like obesity, sarcopenic-obesity, sarcopenia, diabetes and heart failure and especially compounds that retains the selective activation of the CRF₂ receptor, while having improved properties on other parameters, e.g. longer half-lifes, improved stability or other improved properties.

Patent applications disclosing different derivatives of Ucn2 and their potential use in medicine are disclosed in e.g. EP2470560, WO08047241 and WO18013803.

### SUMMARY

The present invention relates to Ucn2 derivatives which selectively activates the human CRF₂ receptor and which have high chemical and physical stability, suitable for subcutaneous, e.g. once weekly, administration to humans. This is achieved by the combination of certain peptides that are analogues of Ucn2 with fatty acid-based substituents.

In a first aspect the invention relates to a Ucn2 derivative comprising a Ucn2 analogue and a fatty acid substituent. The Ucn2 derivative is a CRF₂ receptor agonist selective towards the CRF₂ receptor over the CRF₁ receptor. The Ucn2 derivative has a surprisingly high physical stability in a liquid formulation. Also or alternatively, the Ucn2 derivative has a surprisingly high chemical stability in a liquid formulation.

In a further aspect the invention relates to a compound that has a terminal half-life suitable for once-weekly administration in human. In a further aspect, the invention relates to compound which is a Ucn2 derivative that upon administration into the body reduces body weight and/or reduces body fat mass and/or improves body composition. Also or alternatively it increases muscle mass and/or muscle function. Also or alternatively it lowers food intake. Also or alternatively it improves insulin/glucose parameters. In a further aspect the invention relates to use of the compounds described herein for use as a medicament. Also or alternatively the invention relates to use of the compounds described herein for use in the prevention or treatment of type 2 diabetes. Also or alternatively the invention relates to use of the compounds described herein for use in the prevention or treatment of obesity. Also or alternatively the invention relates to use of the compounds described herein for use in the prevention or treatment of cardiovascular disease. Also or alternatively the invention relates to use of the compounds described herein for use in the prevention or treatment of heart failure. Also or alternatively the invention relates to use of the compounds described herein for use in the prevention or treatment of sarcopenia.

In a further aspect the invention relates to a method of preventing or treating type 2 diabetes by administering the Ucn2 derivative of the invention to a patient in need thereof. Also or alternatively the invention relates to a method of preventing or treating obesity by administering a compound described herein to a patient in need thereof. Also or alternatively the invention relates to a method of preventing or treating cardiovascular disease by administering a compound described herein to a patient in need thereof. Also or alternatively the invention relates to a method of preventing or treating heart failure by administering a compound described herein to a patient in need thereof. Also or alternatively the invention relates to a method of preventing or treating sarcopenia by administering a compound described herein to a patient in need thereof.

In a further aspect, the invention relates to a pharmaceutical composition comprising the Ucn2 derivative of the invention.

### BRIEF DESCRIPTION OF DRAWINGS

FIG. 1: Physical stability of the compounds of the invention in a liquid formulation measured as change in turbidity reported as average pixel intensity over time.

FIG. 2: Physical stability of the compounds of the invention in a liquid formulation measured as change in amount of visible particles reported as particle density over time.

### DESCRIPTION

In what follows, Greek letters may be represented by their symbol or the corresponding written name, for example: α = alpha; β = beta; ε = epsilon; γ = gamma; ω = omega; etc. Also, the Greek letter of µ may be represented by “u”, e.g. in µL = uL, or in µM = uM.

In one aspect, the compounds of the invention comprises a peptide of the sequence:K-I-V-L-S-L-D-V-P-I-G-L-L-Q-I-L-L-E-Q-A-R-A-R-A-A-R-E-Q-A-T-T-N-A-E-I-L-X₃₆-X₃₇-V; wherein X₃₆ is A or E, wherein X₃₇ is R or E; wherein at least one of X₃₆ and X₃₇ is E; and wherein a substituent is attached to the N-terminal K of the peptide; or a pharmaceutically acceptable salt thereof.

### Compound/Product

### CRF₂ Receptor Agonist

A receptor agonist may be defined as a compound that binds to a receptor and elicits a response typical of the natural ligand (see e.g. “Principles of Biochemistry ”, AL Lehninger, DL Nelson, MM Cox, Second Edition, Worth Publishers, 1993, page 763). Thus, for example, a “CRF₂ receptor agonist” may be defined as a compound which is capable of binding to the CRF₂ receptor and capable of activating it.

### Ucn2 Analogue

The term “hUcn1” as used herein refers to the human Urocortin 1 peptide, the sequence which is included in the sequence listing as SEQ ID NO: 1. The term “hUcn2” as used herein refers to the human Urocortin 2 peptide, the sequence which is included in the sequence listing as SEQ ID NO: 2. The peptide having the sequence of SEQ ID NO: 2 may also be designated native Ucn2. The peptide sequence of hUcn2 has an amide group at the C-terminus. If a peptide has an amide group at the C-terminus, the C-terminus is said to be “amidated”, and thus the C-terminus of hUcn2 is amidated.

The term “Ucn2 analogue” as used herein refers to a variant of hUcn2 wherein a number of amino acid residues has been changed as compared to hUcn2 and wherein the ability to selectively activate the human CRF₂ receptor (over the human CRF₁ receptor) has been retained. These amino acid changes may represent, independently, one or more amino acid substitutions, additions, and/or deletions. Amino acid residues may be identified by their full name, their one-letter code, and/or their three-letter code. These three ways are fully equivalent.

The term “substitution” as used herein in context of a peptide sequence refers to the replacement of one amino acid of peptide sequence with another amino acid. In one aspect, amino acids may be substituted by conservative substitution. The term “conservative substitution” as used herein denotes that one or more amino acids are replaced by another, biologically similar residue. Examples include substitution of amino acid residues with similar characteristics, e.g. small amino acids, acidic amino acids, polar amino acids, basic amino acids, hydrophobic amino acids and aromatic amino acids.

Ucn2 analogues of the compounds of the invention may be described by reference to i) the number of the amino acid residue in hUcn2 which corresponds to the amino acid residue which is changed (i.e., the corresponding position in hUcn2, and to ii) the actual change. For example, [Lys24]-hUcn2 refers to a Ucn2 analogue in which position 24 of hUcn2 has been replaced by a Lys.

An example of nomenclature used herein is Lys-[Glu33,Glu37]-hUcn2, in which the hUcn2 analogue comprises three amino acid changes as compared to hUcn2, namely a Lys residue addition in position -1 (also referred to as “N-terminal K”, corresponding to an N-terminal elongation), a Glu substitution in position 33 as well as a Glu substitution in position 37. In the sequence listing, the first amino acid starting from the N-terminus (i.e. the N-terminal K) is assigned no. 1.

The term “peptide”, as used herein, refers to a compound which comprises a series of amino acids interconnected by amide (or peptide) bonds. Amino acids are molecules containing an amino group and a carboxylic acid group, and, optionally, one or more additional groups, often referred to as a side chain.

The term “amino acid” includes proteinogenic (or coded or natural) amino acids (amongst those the 20 standard amino acids), as well as non-proteinogenic (or non-coded or non-natural) amino acids. Proteinogenic amino acids are those which are naturally incorporated into proteins. The standard amino acids are those encoded by the genetic code. Non-proteinogenic amino acids are either not found in proteins, or not produced by standard cellular machinery (e.g., they may have been subject to post-translational modification). In what follows, all amino acids of the Ucn2 analogues and compounds of the invention for which the optical isomer is not stated is to be understood to mean the L-isomer (unless otherwise specified).

In one embodiment the compounds of the invention comprises a Ucn2 analogue selected from a list of SEQ ID NO: 3 and SEQ ID NO: 4. In one embodiment the C-terminus of the Ucn2 analogue is optionally amidated. In one preferred embodiment the C-terminus of the Ucn2 analogue is amidated.

### Ucn2 Derivatives

The compounds of the invention are Ucn2 derivatives. The term “derivative” as used herein means a chemically modified Ucn2 analogue, in which one or more substituents have been covalently attached to the peptide backbone. The substituent may also be referred to as a side chain. In one aspect, the Ucn2 derivative comprises a substituent covalently attached to the analogue described herein via a Lys (K) residue in position -1. In one embodiment, the Ucn2 derivative comprises a substituent covalently attached to the epsilon amino group of the Lys at position -1.

In some embodiments, the Ucn2 derivative comprises or consists of a substituent as defined below covalently linked to a Ucn2 analogue. Such compounds may be referred to as derivatives of the peptide or derivatives of the Ucn2 analogue or simply derivatives, as they are obtained by covalently linking a substituent to a Ucn2 analogue peptide backbone.

In one embodiment the Ucn2 derivative comprises a peptide of the sequence: K-I-V-L-S-L-D-V-P-I-G-L-L-Q-I-L-L-E-Q-A-R-A-R-A-A-R-E-Q-A-T-T-N-A-E-I-L-X₃₆-X₃₇-V; wherein X₃₆ is A or E, wherein X₃₇ is R or E; wherein at least one of X₃₆ and X₃₇ is E; and wherein a substituent is attached to the peptide via the N-terminal K; or a pharmaceutically acceptable salt thereof. In one embodiment only one of X₃₆ and X₃₇ is E. In one embodiment X₃₆ is E and X₃₇ is R. In one embodiment X₃₆ is A and X37 is E. In a most preferred embodiment, the compound of the invention is selected from a list consisting of Compound 3 and Compound 4.

### Substituent

The term “substituent” as used herein in context of the Ucn2 derivative refers to a chemical moiety or group which replaces a hydrogen atom of an amino acid residue of the Ucn2 analogue. The substituent may be attached to the Ucn2 analogue by acylation, i.e. via an amide bond formed between a carboxylic acid group of the substituent and e.g. the lysine at position -1 of the Ucn2 analogue. In one embodiment, the substituent is attached via the epsilon-amino group of a Lys at position -1 of the Ucn2 analogue. In one embodiment, the substituent is attached to the analogue at the N-terminus of the Ucn2 analogue. In one aspect of the invention, the substituent may be an N-terminal substituent. The term “N-terminal substituent” as used herein, means a chemical moiety or group replacing a hydrogen atom at the amino acid residue which is located at the N-terminal position of an amino acid sequence. In one embodiment, the substituents described herein are covalently attached to the Ucn2 analogue of the compounds of the invention via a Lys residue in position -1. The Lys residue in position -1 of the Ucn2 analogues of the compounds of the invention may also be referred to as the “N-terminal K”. In one embodiment, the substituent is attached to the Ucn2 analogue via the epsilon-amino group of a Lys when said Lys is included at position -1. In one embodiment, the substituent is attached to the Ucn2 analogue at the N-terminus of the peptide. In one embodiment, the substituent is covalently attached to a Lys residue of the Ucn2 analogue by acylation, i.e. via an amide bond formed between a carboxylic acid group of the substituent and the epsilon-amino group of the Lys residue.

The substituent may comprise several elements, such as a protractor element (referred to as Prot) and one or more linker elements (referred to as Z1 and Z2). In some embodiments, the protractor is linked to the linker elements by amide bonds (see further below). As further defined herein, below the number of linker elements may be referred to as *-Z1-Z2-* where Z1 is connected to the protractor (Prot) and Z2 is connected to the Ucn2 analogue, in which case the substituent may be referred to as Prot-Z1-Z2-*.

In one embodiment, the substituent comprises at least one protractor. In one embodiment, the term “protractor” is used to describe the fatty acid group which is the terminal part of the substituent responsible for extending half-life of the compound. In one embodiment, the protractor is a fatty acid group. In such an embodiment, the fatty acid group comprises a carbon chain which contains at least 8 consecutive —CH₂— groups. In one embodiment, the fatty acid group comprises 8-20 consecutive —CH₂— groups. In one embodiment, the fatty acid group comprises 10-18 consecutive —CH₂— groups. In one embodiment, the fatty acid group comprises 12-18 consecutive —CH₂— groups. In one embodiment, the fatty acid group comprises 14-18 consecutive —CH₂— groups.

In one embodiment, the protractor (Prot) is of the Formula: HOOC—(CH₂)ₙ—CO—* wherein n is an integer in the range of 8-20. This moiety which may also be referred to as a C(n+2) diacid, wherein n is an integer in the range of 8-20 or as

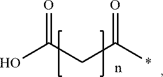

wherein n is an integer in the range of 8-20.

In one embodiment the protractor (Prot) is Chem. 1. In one embodiment, n is an integer in the range of 16-18. In a most preferred embodiment, n is 18. In one embodiment, the protractor is a diacid. In a preferred embodiment, the protractor is a C18-C20 diacid. In a most preferred embodiment, the protractor is a C20 diacid.

The symbol “*” generally indicates an attachment point, thus the above * indicates the attachment point of the protractor to Z1, which when bound via an amide bond is a nitrogen. In one embodiment, the substituent is defined by: Prot-Z1-Z2- wherein Prot- is Chem. 1, and wherein n is an integer in the range of 16-20. In a particular embodiment, n is 14, 16, 18 or 20 in Chem. 1. In a particular embodiment, n is 16, 18 or 20 in Chem. 1. In a particular embodiment, n is 18 or 20 in Chem. 1. In a particular embodiment, the protractor (Prot) is a C18-C20 diacid. In a particular embodiment, the protractor (Prot) is a C18 diacid. In a particular embodiment, the protractor (Prot) is a C20 diacid.

The linker elements Z1 and Z2 are individually selected from chemical moieties capable of forming amide bonds. In some embodiments, the linker elements Z1 and Z2 are individually selected from chemical moieties such as gGlu (also termed gamma Glu or γGlu) or Ado (also termed or 8-amino-3,6-dioxaoctanoic acid).

gGlu is defined by the formula: *—NH—CH(COOH)—(CH₂)₂—CO—* and may also be described by

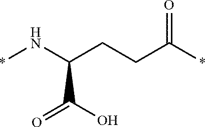 [SMILES: *N[C@@H](CCC(*)=O)C(=O)O]

Ado may be referred to as 8-amino-3,6-dioxaoctanoic acid and is defined by the formula: *—NH—(CH₂)₂—O—(CH₂)₂—O—CH₂—CO—* and may also be described by

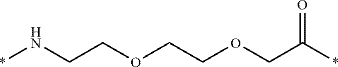 [SMILES: *NCCOCCOCC(*)=O]

In one embodiment, Z1 is selected from one to three gGlu, In one preferred embodiment, Z1 is 3xgGlu. In one embodiment, Z2 is one to two Ado. In a preferred embodiment, Z2 is 2xAdo.

In a most preferred embodiment the substituent is of Formula: Prot-Z1-Z2-*, wherein Prot- is Chem. 1, wherein n of Chem. 1 is 18, wherein Z1 is 3xgGlu, and wherein Z2 is 2xAdo. This substituent is also described by

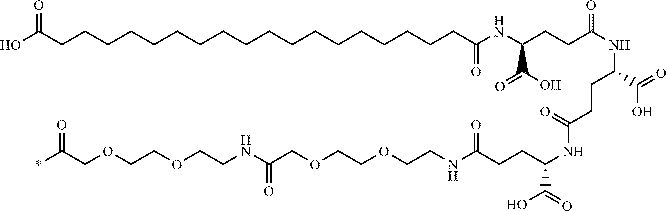 [SMILES: *C(=O)COCCOCCNC(=O)COCCOCCNC(=O)CC[C@H](NC(=O)CC[C@H](NC(=O)CC[C@H](NC(=O)CCCCCCCCCCCCCCCCCCC(=O)O)C(=O)O)C(=O)O)C(=O)O]

The compounds of the invention may exist in different stereoisomeric forms having the same molecular formula and sequence of bonded atoms but differing only in the three-dimensional orientation of their atoms in space. Unless otherwise stated the invention relates to all stereoisomeric forms of the embodied compound.

### Pharmaceutically Acceptable Salts

The compounds of the invention may be in the form of a pharmaceutically acceptable salt or amide. Salts are e.g. formed by a chemical reaction between a base and an acid, e.g.: 2NH₃ + H₂SO₄ → (NH₄)₂SO₄. The salt may be a basic salt, an acid salt, or it may be neither (i.e. a neutral salt). Basic salts produce hydroxide ions and acid salts hydronium ions in water. The salts of the compounds of the invention may be formed with added cations or anions between anionic or cationic groups, respectively. These groups may be situated in the peptide moiety and/or in the substituent of the compounds of the invention. Non-limiting examples of anionic groups include any free carboxylic acid groups in the substituent, if any, as well as in the peptide moiety. The peptide moiety may include a free carboxylic acid group at the C-terminus, if present, as well as any free carboxylic acid group of internal acidic amino acid residues such as Asp and Glu. Non-limiting examples of cationic groups include any free amino groups in the substituent, if any, as well as in the peptide moiety. The peptide moiety may include a free amino group at the N-terminus, if present, as well as any free amino group of internal basic amino acid residues such as His, Arg and Lys. The amide of the compounds of the invention may, e.g., be formed by the reaction of a free carboxylic acid group with an amine or a substituted amine, or by reaction of a free or substituted amino group with a carboxylic acid. The amide formation may involve the free carboxylic group at the C-terminus of the peptide, any free carboxylic group in the side chain, the free amino group at the N-terminus of the peptide, and/or any free or substituted amino group of the peptide in the peptide and/or the side chain. In one aspect, compounds of the invention are in the form of a pharmaceutically acceptable salt. In a further aspect, the Ucn2 analogue or Ucn2 derivative is in the form of a pharmaceutically acceptable salt. Also or alternatively, in one aspect, the derivative is in the form of a pharmaceutically acceptable amide, preferably with an amide group at the C-terminus of the peptide.

### Functional Properties

In a first functional aspect, the compounds of the invention have a good binding affinity to and a good potency at the human CRF₂ receptor. The compounds of the invention are also selective towards the human CRF₂ receptor over the human CRF₁ receptor. Also, or alternatively, in a second functional aspect, they have an in vivo effect on body weight, body composition, food intake and glucose tolerance both alone and in combination with a GLP-1 receptor agonist. Also, or alternatively, in a third functional aspect, they have improved pharmacokinetic properties. Also, or alternatively, in a fourth functional aspect, the compounds of the invention are physically stable. Also, or alternatively, in a fifth functional aspect, the compounds of the invention are chemically stable.

### Biological Activity - In Vitro Binding and Potency

According to a functional aspect, the compounds of the invention are biologically active, i.e. capable of binding (i.e. binding affinity) the human CRF₂ receptor and/or potent at the human CRF₂ receptor. Also or alternatively the compound is selective toward the human CRF₂ receptor over the human CRF₁ receptor.

In one embodiment the binding affinity of the human CRF₂ receptor refers to the ability to bind the receptor in vitro. In one embodiment the binding is measured as described in Example 2. The binding affinity may be expressed by the IC₅₀ value. In one embodiment the compounds of the invention binds the human CRF₂ with an IC50 of <100 nM, preferably an IC50 of <50 nM, more preferably an IC50 of <10 nM, most preferably an IC50 of <5 nM, when measured as described in Example 2.

In one embodiment, potency refers to the ability to activate the CRF₂ receptor in vitro. In one embodiment the activation is measured as described in Example 3. The potency may be expressed by EC₅₀ value. In one embodiment the compounds of the invention activates the human CRF₂ with an EC50 of <200 nM, preferably an EC50 of <100 nM, more preferably an EC50 of <75 nM, most preferably an EC50 of <60 nM, when measured as described in Example 3.

### Biological Activity - In Vivo Pharmacology

According to a second functional aspect of the invention, the compounds of the invention are potent in vivo, which may be determined as is known in the art in any suitable animal model, as well as in clinical trials. One example of a suitable animal model is ad libitum fed rats, and the effect of a single subcutaneous dose of the compounds on food intake in the rats over at least two days after dosing can be assessed in this model, e.g. as illustrated in Example 5 herein. The compounds of the invention are very potent in vivo, which is evidenced by a relevant reduction in food intake in this study in ad libitum fed rats. The diet-induced obese (DIO) rat is another example of a suitable animal model. The effect of the compounds of the invention on food intake, body weight, body composition, oral glucose tolerance, muscle mass and biomarkers after sub-chronic dosing for 3 weeks can be measured in such DIO rats in vivo, e.g. as described in Example 6 herein, where the compounds of the invention showed improvement in body composition and in parameters related to glucose metabolism.

### Pharmacokinetics Profile - Half Life In Vivo In Minipigs

According to the third functional aspect, the compounds of the invention have desirable pharmacokinetic properties such as increased terminal half-life as compared to human Ucn2. Desirable terminal half-life means that the compound in question is eliminated from the body at a rate that makes it suitable e.g. for weekly administration. For the compounds of the invention this entails an extended duration of pharmacological effect. The pharmacokinetic properties of the compounds of the invention may suitably be determined in vivo in pharmacokinetic (PK) studies. Such studies are conducted to evaluate how pharmaceutical compounds are absorbed, distributed, and eliminated in the body, and how these processes affect the concentration of the compound in the body, over the course of time. In the discovery and preclinical phase of pharmaceutical drug development, animal models such as the mouse, rat, monkey, dog, or pig, may be used to perform this characterisation. Any of these models can be used to test the pharmacokinetic properties of the compounds of the invention. In such studies, animals are typically administered with a single dose of the drug, either intravenously (i.v.), subcutaneously (s.c.), or orally (p.o.) in a relevant formulation. Blood samples are drawn at predefined time points after dosing, and samples are analysed for concentration of drug with a relevant quantitative assay. Based on these measurements, plasma concentration vs. time profiles for the compound of study are plotted and a so-called non-compartmental pharmacokinetic analysis of the data is performed. For most compounds, the terminal part of the plasma-concentration profiles will be linear when drawn in a semi-logarithmic plot, reflecting that after the initial absorption and distribution, drug is removed from the body at a constant fractional rate. The rate (lambda Z or Iz) is equal to minus the slope of the terminal part of the plot. From this rate, also a terminal half-life may be calculated, as t½= In(2) / Iz (see, e.g., Johan Gabrielsson and Daniel Weiner: Pharmacokinetics and Pharmacodynamic Data Analysis. Concepts & Applications, 3rd Ed., Swedish Pharmaceutical Press, Stockholm (2000)). Clearance can be determined after i.v. administration and is defined as the dose (D) divided by area under the curve (AUC) of the plasma concentration versus time profile (Rowland, M and Tozer TN: Clinical Pharmacokinetics: Concepts and Applications, 3rd edition, 1995 Williams Wilkins). The estimate of terminal half-life and/or clearance is relevant for evaluation of dosing regimens and an important parameter in drug development, in the evaluation of new drug compounds.

According to the third functional aspect, the compounds of the invention have desirable pharmacokinetic properties. In a particular embodiment, the pharmacokinetic properties may be determined as terminal half-life (t½) in vivo in minipigs after i.v. administration, e.g. as described in Example 4. In one embodiment wherein the compound of the invention has a terminal half-life of >50 hours, preferably a terminal half-life of >60 hours, more preferably a terminal half-life of >70 hours, even more preferably a terminal half-life of >80 hours, and most preferably a terminal half-life of >90 hours, when measured as described in Example 4

### Physical Properties

According to the fourth functional aspect, the compounds of the invention are associated with a high physical stability in a pharmaceutical solution. The term “physical stability” refers to the tendency of the compound to form biologically inactive and/or insoluble aggregates (such as visible particles) in a liquid formulation or a gel. In a particular embodiment, the physical stability of the compounds of the invention is assessed by measuring formation of visible particles or change in turbidity over time, e.g. as described in Example 7.

In one embodiment the compounds of the invention have high physical stability when assessed by measuring formation of visible particles over time in a liquid formulation as described in Example 7, wherein no visible particles is detected after 14 days.

### Chemical Properties

According to the fifth functional aspect, the compounds of the invention have high chemical stability. The term “chemical stability” refers to chemical (in particular covalent) changes in the compound leading to formation of chemical degradation products, such as high molecular weight proteins (HMWPs), deamidation, isomerization and hydrolysis products potentially having a reduced biological potency, and/or increased immunogenic effect as compared to the intact polypeptide. In a particular embodiment, the improved chemical stability may be determined by measuring the purity loss, i.e. by measuring the amount of chemical degradation to the compound at various time-points after exposure to different environmental conditions, e.g. by size exclusion liquid chromatography, reversed-phase liquid chromatography and/or liquid chromatography coupled to mass spectrometry, e.g. as described in Example 8 herein.

In one embodiment the compounds of the invention have an average purity loss per week of <6.00%, preferably an average purity loss per week of <5.00%, more preferably an average purity loss per week of <4.00%, even more preferably an average purity loss per week of <3.00%, and most preferably an average purity loss per week of <2.00%, when measured as described in Example 8.

### Production Processes

The production of peptides like Ucn2 analogues and derivatives is well known in the art. The compounds of the invention (or fragments thereof), may be prepared by classical peptide synthesis, e.g. solid phase peptide synthesis using t-Boc or Fmoc chemistry or other well established techniques, see e.g. Greene and Wuts, “Protective Groups in Organic Synthesis”, John Wiley & Sons, 1999, Florencio Zaragoza Dörwald, “Organic Synthesis on solid Phase”, Wiley-VCH Verlag GmbH, 2000, and “Fmoc Solid Phase Peptide Synthesis”, Edited by W.C. Chan and P.D. White, Oxford University Press, 2000. Also or alternatively, the compounds of the invention (or fragments hereof) may be produced, in whole or in part, by recombinant methods, viz. by culturing a host cell containing a DNA sequence encoding the analogue and capable of expressing the peptide in a suitable nutrient medium under conditions permitting the expression of the peptide. Non-limiting examples of host cells suitable for expression of these peptides are Escherichia coli and Saccharomyces cerevisiae, as well as mammalian BHK or CHO cell lines.

The compounds of the present invention may be purified by a variety of procedures known in the art including, but not limited to, chromatography (e.g., ion exchange, affinity, hydrophobic, and reversed-phase high performance liquid chromatography), electrophoretic procedures, or extraction (see, e.g., Protein Purification, J.-C. Janson and Lars Ryden, editors, VCH Publishers, New York, 1989). Specific examples of methods of preparing compounds of the invention are included in Example 1.

### Pharmaceutical Compositions

In a further aspect the invention relates to a pharmaceutical composition comprising the compounds of the invention. Injectable compositions comprising compounds of the invention can be prepared using the conventional techniques of the pharmaceutical industry which involve dissolving and mixing the ingredients as appropriate to give the desired end product. Thus, according to one procedure, a compound of this invention is dissolved in a suitable buffer at a suitable pH so precipitation is minimised or avoided. The injectable composition is made sterile, for example, by sterile filtration. Pharmaceutical compositions comprising a compound of the invention or a pharmaceutically acceptable salt, or amide thereof, and a pharmaceutically acceptable excipient may be prepared as is known in the art. The term “excipient” broadly refers to any component other than the active therapeutic ingredient(s). The excipient may be an inert substance, an inactive substance, and/or a not medicinally active substance. The formulation of pharmaceutically active ingredients with various excipients is known in the art, see e.g. Remington: The Science and Practice of Pharmacy (e.g. 19th edition (1995), and any later editions). A composition may be a stabilised formulation. The term “stabilised formulation” refers to a formulation with increased physical and/or chemical stability, preferably both. In general, a formulation must be stable during use and storage (in compliance with recommended use and storage conditions) until the expiration date is reached.

### Combination Treatments

The treatment with a compound of the invention may also be combined with one or more additional pharmacologically active substances, e.g. selected from antidiabetic agents, antiobesity agents, appetite regulating agents, antihypertensive agents, agents for the treatment and/or prevention of complications resulting from or associated with diabetes and agents for the treatment and/or prevention of complications and disorders resulting from or associated with obesity. Examples of these pharmacologically active substances are: GLP-1 (glucagon-like peptide-1) receptor agonists, insulin, DPP-IV (dipeptidyl peptidase-IV) inhibitors, amylin agonists, GIP (glucose-dependent insulinotropic polypeptide) receptor agonists and leptin receptor agonists.

In one aspect of the invention, a Ucn2 derivative according to the present invention is combined with a GLP-1 agonist. The compounds may be supplied in a single dosage form wherein the single-dosage form contains both compounds, or in the form of a kit-of-parts comprising a preparation of the Ucn2 analogue as a first unit dosage form and a preparation of the GLP-1 agonist as a second unit dosage form. Non-limiting examples of GLP-1 agonists to be combined with the Ucn2 analogues of the present invention are liraglutide, semaglutide, exenatide, dulaglutide, lixisenatide, taspoglutide, and albiglutide. Semaglutide is a GLP-1 receptor agonist that may be prepared as described in WO2006/097537, Example 4 and is also known as N6,26-{18-[N-(17-carboxyheptadecanoyl)-L-γ-glutamyl]-10-oxo-3,6,12,15-tetraoxa-9,18-diazaoctadecanoyl}-[8-(2-amino-2-propanoic acid), 34-L-arginine]human glucagon-like peptide 1(7-37), see WHO Drug Information Vol. 24, No. 1, 2010 (SEQ ID NO: 57). Liraglutide, a mono-acylated GLP-1 derivative for once daily administration which is marketed under the brand name Victoza® by Novo Nordisk A/S, is disclosed in WO 98/08871. WO 2006/097537 discloses GLP-1 derivatives including semaglutide, a mono-acylated GLP-1 derivative for once weekly administration marketed under the brand name Ozempic® by Novo Nordisk A/S.

### Pharmaceutical Indications

A further aspect of the invention relates to the use of compounds of the invention as a medicament. Compositions comprising the compounds or a pharmaceutically acceptable salt hereof, and optionally one or more a pharmaceutically acceptable excipients may be prepared as is known in the art. In particular embodiments, the compounds of the invention described herein may be used in the following medical treatments:
(i) prevention and/or treatment of all forms of diabetes, such as hyperglycaemia, type 2 diabetes, impaired glucose tolerance, type 1 diabetes, non-insulin dependent diabetes, MODY (maturity onset diabetes of the young), gestational diabetes, and/or for reduction of HbA1C;
(ii) delaying or preventing diabetic disease progression, such as progression in type 2 diabetes, delaying the progression of impaired glucose tolerance (IGT) to insulin requiring type 2 diabetes, delaying or preventing insulin resistance, and/or delaying the progression of non-insulin requiring type 2 diabetes to insulin requiring type 2 diabetes;
(iii) prevention and/or treatment of eating disorders, such as obesity, e.g. by decreasing food intake, reducing body weight and/or fat mass, improving body composition, suppressing appetite, inducing satiety; treating or preventing binge eating disorder, bulimia nervosa, and/or obesity induced by administration of an antipsychotic or a steroid; reduction of gastric motility; delaying gastric emptying; increasing physical mobility; and/or prevention and/or treatment of comorbidities to obesity, such as osteoarthritis and/or urine incontinence;
(iv) weight maintenance after successful weight loss (either drug induced or by diet and exercise) - i.e. prevention of weight gain after successful weight loss.
(v) prevention and/or treatment of sarcopenic obesity
(vi) prevention and/or treatment of Sarcopenia and/or muscle atrophy of other cause.
(vii) prevention and/or treatment of cardiovascular disease.
(viii) prevention and/or treatment of heart failure.
(ix) prevention and/or treatment of hypertension.
(x) prevention and/or treatment of dyslipidaemia and/or atherosclerosis.
(xi) prevention and/or treatment of NAFLD/NASH
(xii) prevention and/or treatment of chronic kidney disease caused by e.g. hypertension or diabetes.
(xiii) prevention and/or treatment of Cachexia.
(xiv) prevention and/or treatment of neurodegenerative diseases, e.g. Alzheimer’s disease and Parkinson.
(xv) prevention and/or treatment of neuromuscular diseases.
(xvi) prevention and/or treatment of Sleep apnoea, hip/knee osteoarthritis.

In one embodiment, the compounds of the invention are for use in a method for prevention and/or treatment of diabetes and/or obesity. In one embodiment, the compounds are for use in a method for treatment of diabetes and/or obesity. In one embodiment, the compounds are for use in a method for treatment or prevention of type 2 diabetes. In one embodiment, the compounds are for use in a method for treatment of type 2 diabetes. In one embodiment, the compounds are for use in a method for treatment or prevention of obesity. In one embodiment, the compounds are for use in a method for treatment of obesity. In one embodiment, the compounds are for use in a method for weight management. In one embodiment, the compounds are for use in a method for reduction of appetite. In one embodiment, the compounds are for use in a method for reduction of food intake.

Generally, all subjects suffering from obesity are also considered to be suffering from overweight. In some embodiments the invention relates to a method for treatment or prevention of obesity. In some embodiments the invention relates to use of the compounds of the present invention for treatment or prevention of obesity. In some embodiments the subject suffering from obesity is human, such as an adult human or a paediatric human (including infants, children, and adolescents). Body mass index (BMI) is a measure of body fat based on height and weight. The formula for calculation is BMI = weight in kilograms/(height in meters)². A human subject suffering from obesity may have a BMI of ≥30; this subject may also be referred to as obese. In some embodiments the human subject suffering from obesity may have a BMI of ≥35 or a BMI in the range of ≥30 to <40. In some embodiments the obesity is severe obesity or morbid obesity, wherein the human subject may have a BMI of ≥40. In some embodiments the invention relates to a method for treatment or prevention of overweight, optionally in the presence of at least one weight-related comorbidity. In some embodiments the invention relates to use of the compounds of a Ucn2 analogue for treatment or prevention of overweight, optionally in the presence of at least one weight-related comorbidity. In some embodiments the subject suffering from overweight is human, such as an adult human or a paediatric human (including infants, children, and adolescents). In some embodiments a human subject suffering from overweight may have a BMI of ≥25, such as a BMI of ≥27. In some embodiments a human subject suffering from overweight has a BMI in the range of 25 to <30 or in the range of 27 to <30. In some embodiments the weight-related comorbidity is selected from the group consisting of hypertension, diabetes (such as type 2 diabetes), dyslipidaemia, high cholesterol, and obstructive sleep apnoea. In some embodiments the invention relates to a method for reduction of body weight. In some embodiments the invention relates to use of the compounds of the invention for reduction of body weight. A human to be subjected to reduction of body weight according to the present invention may have a BMI of ≥25, such as a BMI of ≥27 or a BMI of ≥30. In some embodiments the human to be subjected to reduction of body weight according to the present invention may have a BMI of ≥35 or a BMI of ≥40. The term “reduction of body weight” may include treatment or prevention of obesity and/or overweight. Reduction of body weight may also include reduction in fat mass.

### EMBODIMENTS

1. A compound comprising a peptide of the sequence:
   K-I-V-L-S-L-D-V-P-I-G-L-L-Q-I-L-L-E-Q-A-R-A-R-A-A-R-E-Q-A-T-T-N-A-E-I-L-X₃₆-X₃₇-V; wherein
   X₃₆ is A or E,
   X₃₇ is R or E;
   wherein at least one of X₃₆ and X₃₇ is E;
   wherein a substituent is attached to the peptide via the N-terminal K;
   or a pharmaceutically acceptable salt thereof.
2. The compound according to any preceding embodiment, with the proviso that at least one of X₃₆ and X₃₇ is E.
3. The compound according to any preceding embodiment, with the proviso that only one of X₃₆ and X₃₇ is E.
4. The compound according to any preceding embodiment, wherein X₃₆ is E and X₃₇ is R.
5. The compound according to any preceding embodiment, wherein X₃₆ is A and X37 is E.
6. The compound according to any preceding embodiment, wherein the C-terminus of the peptide is amidated.
7. The compound according to any preceding embodiment, wherein the substituent is attached via an amide bond to the epsilon amino group of the N-terminal K.
8. The compound according to any preceding embodiment, wherein the substituent is covalently attached to the epsilon amino group of the N-terminal K via an amide bond.
9. The compound according to any preceding embodiment, wherein the substituent comprises at least one protractor (Prot).
10. The compound according to any preceding embodiment, wherein the protractor (Prot) is a fatty acid.
11. The compound according to any preceding embodiment, wherein the protractor (Prot) is a C20 diacid.
12. The compound according to any preceding embodiment, wherein the protractor (Prot) is Chem. 1.
13. The compound according to any preceding embodiment, wherein n of Chem. 1 is an integer in the range 16-18.
14. The compound according to any preceding embodiment, wherein n of Chem. 1 is 18.
15. The compound according to any preceding embodiment, wherein the substituent comprises at least one linker element.
16. The compound according to any preceding embodiment, wherein the substituent comprises three residues of Chem. 2 and two residues of Chem. 3.
17. The compound according to any preceding embodiment, wherein the substituent is of Formula: Prot-Z1-Z2-*, wherein Prot- is Chem. 1, wherein n of Chem. 1 is 18, wherein Z1 is 3xgGlu, and wherein Z2 is 2xAdo.
18. The compound according to any preceding embodiment, wherein the substituent is Chem. 4.
19. The compound according to any preceding embodiment, wherein the compound is a CRF₂ receptor agonist.
20. The compound according to any preceding embodiment, wherein the compound is a CRF₂ derivative.
21. The compound according to any preceding embodiment, wherein the peptide is a CRF₂ analogue.
22. The compound according to any preceding embodiment, which is capable of binding the human CRF₂ receptor.
23. The compound according to any preceding embodiment, which is capable of binding the human CRF₂ receptor as measured in a receptor binding assay as described in Example 2.
24. The compound according to any preceding embodiment, which binds the human CRF₂ with an IC50 of <100 nM, preferably an IC50 of <50 nM, more preferably an IC50 of <10 nM, most preferably an IC50 of <10 nM.
25. The compound according to any preceding embodiment, which binds the human CRF₂ with an IC50 of <100 nM, preferably an IC50 of <50 nM, more preferably an IC50 of <10 nM, most preferably an IC50 of <10 nM; when measured as described in Example 2.
26. The compound according to any preceding embodiment, which is an agonist of the human CRF₂ receptor.
27. The compound according to any preceding embodiment, which is biologically active, or potent at the human CRF₂ receptor.
28. The compound according to any preceding embodiment, which is capable of activating the human CRF₂ receptor.
29. The compound according to any preceding embodiment, which are capable of activating the human CRF₂ receptor as measured in β-Arrestin Assay as described in Example 3.
30. The compound according to any preceding embodiment, which activates the human CRF₂ with an EC50 of <200 nM, preferably an EC50 of <100 nM, more preferably an EC50 of <75 nM, most preferably an EC50 of <60 nM.
31. The compound according to any preceding embodiment, which activates the human CRF₂ with an EC50 of <200 nM, preferably an EC50 of <100 nM, more preferably an EC50 of <75 nM, most preferably an EC50 of <60 nM; when measured as described in Example 3.
32. The compound according to any preceding embodiment, wherein the compound is selective at activating the human CRF₂ receptor over the human CRF₁ receptor.
33. The compound according to any preceding embodiment, wherein the compound has desirable pharmacokinetic properties.
34. The compound according to any preceding embodiment, wherein the compound has long half-life.
35. The compound according to any preceding embodiment, wherein the compound has long terminal half-life when measured in minipigs.
36. The compound according to any preceding embodiment, wherein the compound has a terminal half-life of >50 hours, preferably a terminal half-life of >60 hours, more preferably a terminal half-life of >70 hours, even more preferably a terminal half-life of >80 hours, and most preferably a terminal half-life of >90 hours, when measured as described in Example 4.
37. The compound according to any preceding embodiment, wherein the compound has high physical stability.
38. The compound according to any preceding embodiment, wherein the compound has high physical stability when assessed by measuring formation of visible particles in a liquid formulation over time.
39. The compound according to any preceding embodiment, wherein the compound has high physical stability when assessed by measuring change in turbidity in a liquid formulation over time.
40. The compound according to any preceding embodiment, wherein the compound has high physical stability when assessed by measuring formation of visible particles and/or the change in turbidity over time in a liquid formulation as described in Example 7.
41. The compound according to any preceding embodiment, wherein the compound has high physical stability when assessed by measuring formation of visible particles over time in a liquid formulation as described in Example 7, wherein no visible particles is detected after 14 days.
42. The compound according to any preceding embodiment, wherein the compound has high chemical stability.
43. The compound according to any preceding embodiment, wherein the compound has high chemical stability when assessed by purity loss in a liquid formulation over time.
44. The compound according to any preceding embodiment, wherein the compound has high chemical stability when assessed by purity loss in a liquid formulation over time as described in Example 8.
45. 0
46. The compound according to any preceding embodiment, wherein the compound is selected from the group consisting of Compound 3 and Compound 4.
47. The compound according to any preceding embodiment for use as a medicament.
48. The compound according to any preceding embodiment for use in the prevention and/or treatment of diabetes and/or obesity.
49. The compound according to any preceding embodiment for use in the prevention and/or treatment of cardiovascular disease.
50. A pharmaceutical composition comprising the compound according to any preceding embodiment.
51. The composition according to any preceding embodiment, wherein said composition is an aqueous liquid.
52. The composition according to any preceding embodiment, wherein said composition is an solid composition.
53. A method for prevention and/or treatment of diabetes and/or obesity comprising administering a pharmaceutically active amount of the compound according to any preceding embodiments to a patient in need thereof.
54. A method for prevention and/or treatment of cardiovascular disease comprising administering a pharmaceutically active amount of the compound according to any preceding embodiments to a patient in need thereof.
55. A method for prevention and/or treatment of heart failure comprising administering a pharmaceutically active amount of the compound according to any preceding embodiments to a patient in need thereof.
56. A method for prevention and/or treatment of sarcopenia comprising administering a pharmaceutically active amount of the compound according to any preceding embodiments to a patient in need thereof.
57. A method for preparing a compound according to any of the previous embodiments.

### METHODS AND EXAMPLES

### List of Abbreviations

AUC: Area under the curve
Boc: t-butyloxycarbonyl
BW: Body weight
CAD: Charged Aerosol Detector
CLND: Chemiluminescent Nitrogen Detection
DCM: Dichloromethane
DIC: Diisopropylcarbodiimide
DMF: Dimethylformamide
DMSO: Dimethylsulfoxide
DTT: Dithiothreitol
EDL: Extensor digitorum longus muscle.
Et2O: Diethyl ether
Fmoc: 9-fluorenylmethyloxycarbonyl
GPCR: G-protein coupled receptor
HPLC: High Performance Liquid Chromatography
i.v. intravenous
LC: Liquid Chromatography
LCMS: Liquid Chromatography Mass Spectroscopy
MALDI-MS: Matrix-Assisted Laser Desorption Ionization Mass Spectrometry
MeCN Acetonitrile
MQ: Milli-Q
MS: Mass Spectroscopy
NMP: 1-Methyl-pyrrolidin-2-one
Ado 8-amino-3,6-dioxaoctanoic acid
OGTT: Oral glucose tolerance test
OtBu: tert-butoxy
Oxyma Pure®: Cyano-hydroxyimino-acetic acid ethyl ester
Pbf: 2,2,4,6,7-pentamethyldihydrobenzofuran-5-sulfonyl
p.o. peroral
RP: Reversed Phase
RP-HPLC: Reversed Phase High Performance Liquid Chromatography
RT: Room Temperature
SEC: Size-Exclusion Chromatography
SPA: Scintillation Proximity Assay
SPPS: Solid Phase Peptide Synthesis
tBu: tert-butyl
TFA: trifluoroacetic acid
TIPS: triisopropylsilane
Trt: triphenylmethyl (trityl)
UPLC: Ultra Performance Liquid Chromatography

### General Methods of Preparation

In one aspect the compounds of the invention may be prepared as described in the examples herein. In one aspect the compounds of the invention may be prepared as known in the art, i.e. the preparation of peptides may be produced by classical peptide synthesis, e.g. solid phase peptide synthesis using 180008WO01 33 Boc or Fmoc chemistry or other well-established techniques, see, e.g., Greene and Wuts, “Protective Groups in Organic Synthesis”, John Wiley & Sons, 1999, Florencio Zaragoza Dörwald, “Organic Synthesis on solid Phase”, Wiley-VCH Verlag GmbH, 2000, and “Fmoc Solid Phase Peptide Synthesis”, Edited by W.C. Chan and P.D. White, Oxford University Press, 2000.

This section relates to methods for solid phase peptide synthesis (SPPS methods, including methods for de-protection of amino acids, methods for cleaving the peptide from the resin, and for its purification), as well as methods for detecting and characterising the resulting peptide (LCMS methods).

### Fatty Acid Building Blocks

For synthesis of eicosanedioic acid mono-tert-butyl ester: see WO2010102886 (pages 27-28) for the synthesis of octadecanedioic acid mono-tert-butyl ester. The corresponding mono-tert-butyl ester of C20 diacid can be prepared accordingly.

### Synthesis of C-Terminal Peptide Amides

C-terminal peptide amides were prepared using an amine-based resin (e.g. Fmoc-Rink Amide-MBHA resin, with loading e.g. 0.5 mmol/g). The Fmoc-protected amino acid derivatives used, unless specifically stated otherwise, were the standard recommended— Fmoc—Ala—OH, Fmoc—Arg(Pbf)—OH, Fmoc—Asn(Trt)—OH, Fmoc—Asp(OtBu)—OH, Fmoc—Cys(Trt)—OH, Fmoc—Gln(Trt)—OH, Fmoc—Glu(OtBu)—OH, Fmoc—Gly—OH, Fmoc—His(Trt)—OH, Fmoc—lle—OH, Fmoc—Leu—OH, Fmoc—Lys(Boc)—OH, Fmoc—Met—OH, Fmoc—Phe—OH, Fmoc—Pro—OH, Fmoc—Ser(tBu)—OH, Fmoc—Thr(tBu)—OH, Fmoc—Trp(Boc)—OH, Fmoc—Tyr(tBu)—OH, Fmoc—Val—OH, etc. supplied from e.g. Chemlmpex, Combi-Blocks, Gyros Protein Technologies, Iris Biotech, or Novabiochem. Where nothing else is specified, the natural L-form of the amino acids are used.

In case of modular albumin binding moiety attachment using SPPS, the following suitably protected building blocks such as but not limited to Boc—Lys(Fmoc)—OH, Fmoc-8-amino-3,6-dioxaoctanoic acid (Fmoc—Ado—OH), Fmoc—Glu(OH)—OtBu, and eicosanedioic acid mono-tert-butyl ester were used. All operations stated below were performed within a 0.1-0.2 mmol synthesis scale range.

Unless otherwise specified, SPPS was typically performed using Fmoc based chemistry on a Gyros Protein Technologies SymphonyX solid phase peptide synthesizer, using the manufacturer supplied protocols with minor modifications. Mixing was accomplished by occasional bubbling with nitrogen. The step-wise assembly was performed using the following steps: 1) pre-swelling of resin in DMF; 2) Fmoc-deprotection by the use of 20% (v/v) piperidine in DMF for two treatments of 10 min each; 3) washes with DMF to remove piperidine; 4) coupling of Fmoc-amino acid by the addition of Fmoc-amino acid (5 equivalents) and Oxyma Pure® (5 equivalents) as a 0.5 M solution each in DMF, followed by addition of collidine (10 equivalents) as a 1 M solution in DMF, followed by addition of DIC (5 equivalents) as a 0.5 M solution in DMF, then mixing for 0.5-4 h; 5) draining liquid reagents followed by capping of unreacted peptidyl resin by addition of acetic anhydride (10 equivalents) as a 1 M solution in DMF followed by addition of DMF to reach a final acetic anhydride concentration of 0.25 M; 6) washes with DMF to remove excess reagents; 7) final sequential washes with DCM, MeOH, and Et₂O at the completion of the assembly. Some amino acids such as (but not limited to) Arg or those following a sterically hindered amino acid such as Thr were coupled with an extended reaction time (e.g. 4 h) to ensure reaction completion. Boc—Lys(Fmoc)—OH, Fmoc—Ado—OH, Fmoc—Glu—OtBu, and eicosanedioic acid mono-tert-butyl ester were coupled using the above procedures but with 10 equivalents of each amino acid or carboxylic acid-containing building block as well as 10 equivalents of Oxyma Pure®, and coupling times were extended to 4 h. Reagents such as eicosanedioic acid mono-tert-butyl ester were prepared as a 0.30 M solution in NMP to accommodate the lower solubility in DMF.

### General Cleavage Method

The peptides were cleaved from the polystyrene resin with TFA/TIPS/H2O/Thioanisole/DTT (90:2.5:2.5:2.5:2.5 vol%) for 2 hours, after which the solution was drained into cold diethyl ether and centrifuged. The ether was decanted off, and the peptide was washed thusly with diethyl ether two more times.

### General Method For Purification And Quantification Of The Derivative

The crude peptide was dissolved in water with occasional addition of minimal amounts of MeCN and/or DMSO and/or TFA to facilitate dissolution in a minimum volume, then this solution was filtered through e.g. a 0.2 µm filter. The crude peptides were then purified by reversed-phase preparative HPLC (e.g. Waters Prep 150 LC) on a column comprising C18-silica gel. Elution was performed with an increasing gradient of MeCN in MQ water containing 0.1% TFA. Relevant fractions were analysed with MALDI-MS and UPLC. Fractions containing the pure target peptide were pooled. The resulting solution was analysed (UPLC, LCMS) and the peptide derivative was quantified using a CLND and/or CAD HPLC detector (Ultimate-3000 Thermo-Fischer HPLC connected to an Antek 8060 CLND and/or to a Vanquish CAD). The product was dispensed into glass vials. The vials were capped with Millipore glass fibre prefilters. Freeze-drying afforded the trifluoroacetate salt of the derivative as a white solid.

### General Methods of Detection and Characterisation

This section relates to methods for detection and characterisation of the resulting peptides, including LCMS methods.

General LCMS Method

| | |
|---|---|
| System | LC-system: Waters Acquity UPLC H Class |
| | Column: Waters Acquity UPLC CSH C18 1.7 um, 2.1 x 150 mm |
| | Detector: Waters Xevo G2-XS QTof |
| LC Setup | Eluents: |
| | Solvent A: 99.95% H2O, 0.05% TFA |
| | Solvent B: 99.95% MeCN, 0.05% TFA |
| | Gradient: |
| | 0.0 min: 0.4 mL/min, 5% B, curve initial |
| | 16.0 min: 0.4 mL/min, 95% B, curve 6 |
| | 17.1 min: 0.4 mL/min, 5% B, curve 6 |
| | 20.0 min: 0.4 mL/min, 5% B, curve 6 |
| | 21.0 min: 0.0 mL/min, 5% B, curve 11 |
| | Column temperature: 40 C |
| | Detector: |
| | Channel A: 214 nm |
| | Channel B: 280 nm |
| MS Setup | Source: |
| | ionization mode: ESI+ |
| | analyzer mode: sensitivity |
| | capillary voltage: 3.00 kV |
| | sample cone voltage: 30 V |
| | source temperature: 125 C |
| | desolvation temperature: 400 C |
| | cone gas flow: 50 L/h |
| | desolvation gas flow: 850 L/h |
| | Lockspray: |
| | positive polarity: |
| | reference compound: Leucine Enkephalin |
| | mode: MS |
| | analyzer mode: sensitivity |
| | lock mass: 556.2766 m/z |
| | cone voltage: 30.0 V |
| | capillary: 3.0 kV |
| | DRE transmission: 87.9% |
| | negative polarity: N/A |
| | Collision Cell: |
| | mode: MSe |
| | low energy: 6.00 eV |
| | high energy ramp: 30.00 to 50.00 eV |
| | Detector: |
| | low mass: 75 m/z |
| | high mass: 4000 m/z |
| | scan time: 0.250 s |
| Eluents | Solvent A: 99.95% H2O, 0.05% TFA |
| | Solvent B: 99.95% MeCN, 0.05% TFA |
| | Gradient: |
| | 0.0 min: 0.4 mL/min, 5% B, curve initial |
| | 16.0 min: 0.4 mL/min, 95% B, curve 6 |
| | 17.1 min: 0.4 mL/min, 5% B, curve 6 |
| | 20.0 min: 0.4 mL/min, 5% B, curve 6 |
| | 21.0 min: 0.0 mL/min, 5% B, curve 11 |
| Results | Found mass is the monoisotopic mass found (M/z) of the compound and can include relevant ion adducts such as one or more protons (e.g. +zH) or TFA (+TFA). |
| | Calculated mass is the monoisotopic mass (M/z) calculated for the compound and can include relevant ion adducts such as one or more protons (e.g. +zH) and/or TFA (+TFA). |

### Example 1: Synthesis of Analogues

The Ucn2 analogues and derivatives of the present invention were synthesised according to the general methods of preparation as described above.

### Compound 1 - hUcn1

DNPSLSIDLTFHLLRTLLELARTQSQRERAEQNRIIFDSV-NH₂ (hUcn1)

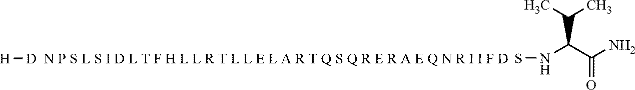

SEQ ID NO: 1

LCMS:
Calculated mass: M/3 = 1565.50; M/4 = 1174.38; M/5 = 939.70; M/6 = 783.25
Found mass: M/3 = 1565.51; M/4 = 1174.39; M/5 = 939.71; M/6 = 783.26

### Compound 2 - hUcn2

IVLSLDVPIGLLQILLEQARARAAREQATTNARILARV-NH₂ (hUcn2)

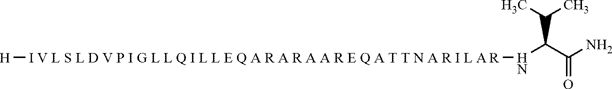

SEQ ID NO: 2

LCMS:
Calculated mass: M/3 = 1384.48; M/4 = 1038.61; M/5 = 831.09
Found mass: M/3 = 1384.50; M/4 = 1038.63; M/5 = 831.11

### Compound 3

N{ε}-[[[(4S)-4-carboxy-4-[[(4S)-4-carboxy-4-[[(4S)-4-carboxy-4-(19-carboxynonadecanoylamino)butanoyl]amino]butanoyl]amino]butanoyl]amino]ethoxy]ethoxy]a cetyl]amino]ethoxy]ethoxy]acetyl]Lys-[Glu33,Glu37]-hUcn2

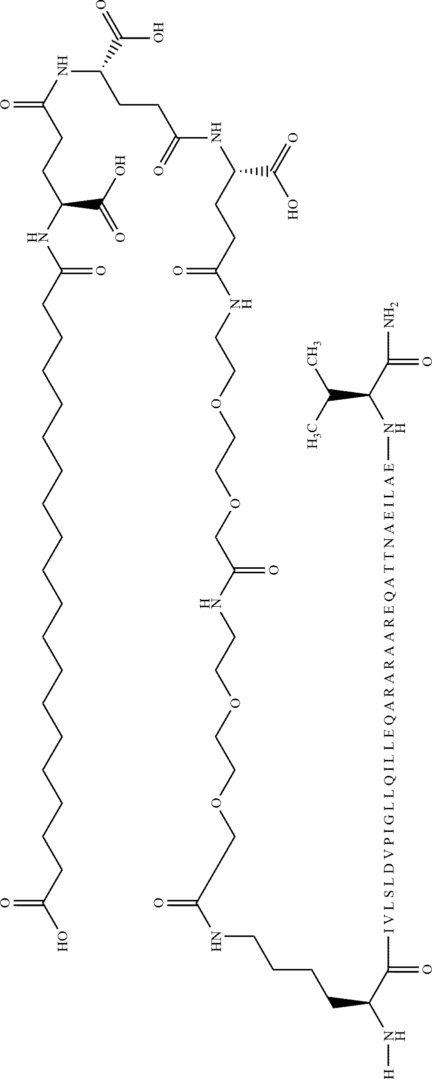

SEQ ID NO: 3

LCMS:
Calculated mass: M/3 = 1742.99; M/4 = 1307.49; M/5 = 1046.19
Found mass: M/3 = 1743.00; M/4 = 1307.48; M/5 = 1046.20

### Compound 4

N{ε}-[[[(4S)-4-carboxy-4-[[(4S)-4-carboxy-4-[[(4S)-4-carboxy-4-(19-carboxynonadecanoylamino)butanoyl]amino]butanoyl]amino]butanoyl]amino]ethoxy]ethoxy]a cetyl]amino]ethoxy]ethoxy]acetyl]Lys-[Glu33,Glu36]-hUcn2

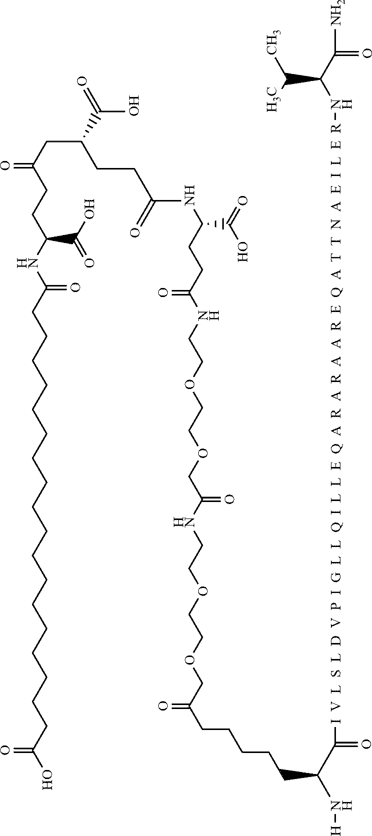

SEQ ID NO: 4

LCMS:
Calculated mass: M/3 = 1771.35; M/4 = 1328.76; M/5 = 1063.21
Found mass: M/3 = 1771.34; M/4 = 1328.76; M/5 = 1063.21

### Compound 5 - Reference Compound

N{ε}-[[[(4S)-4-carboxy-4-[[(4S)-4-carboxy-4-[[(4S)-4-carboxy-4-(19-carboxynonadecanoylamino)butanoyl]amino]butanoyl]amino]butanoyl]amino]ethoxy]ethoxy]a cetyl]amino]ethoxy]ethoxy]acetyl]Lys-hUcn2

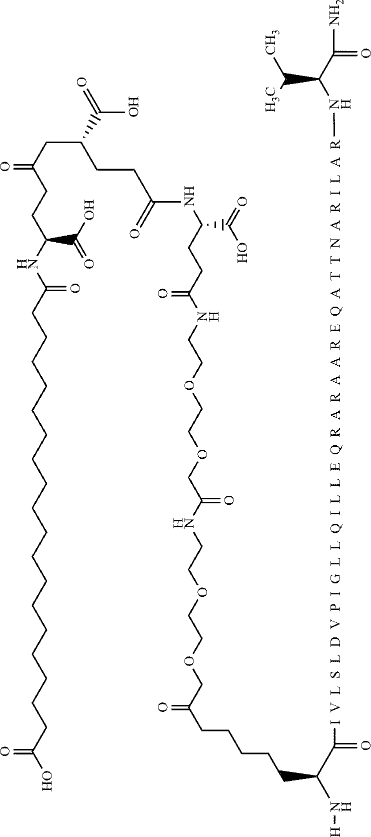

SEQ ID NO: 5

LCMS:
Calculated mass: M/3 = 1761.03; M/4 = 1321.02; M/5 = 1057.02
Found mass: M/3 = 1761.11; M/4 = 1321.07; M/5 = 1057.05

### Compound 6 - Reference Compound

N{ε}-[[[(4S)-4-carboxy-4-[[(4S)-4-carboxy-4-[[(4S)-4-carboxy-4-(19-carboxynonadecanoylamino)butanoyl]amino]butanoyl]amino]butanoyl]amino]ethoxy]ethoxy]a cetyl]amino]ethoxy]ethoxy]acetyl]Lys-[Glu33]-hUcn2

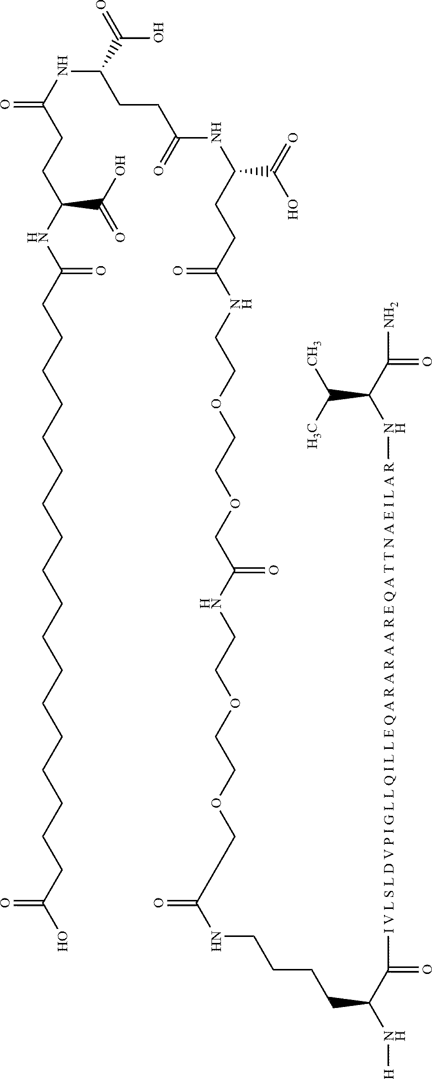

SEQ ID NO: 6

LCMS:
Calculated mass: M/3 = 1752.01; M/4 = 1314.26; M/5 = 1051.61
Found mass: M/3 = 1752.00; M/4 = 1314.26; M/5 = 1051.61

### Compound 7 - Reference Compound

N{ε}-[[[(4S)-4-carboxy-4-[[(4S)-4-carboxy-4-[[(4S)-4-carboxy-4-(19-carboxynonadecanoylamino)butanoyl]amino]butanoyl]amino]butanoyl]amino]ethoxy]ethoxy]a cetyl]amino]ethoxy]ethoxy]acetyl]Lys-[Glu36]-hUcn2

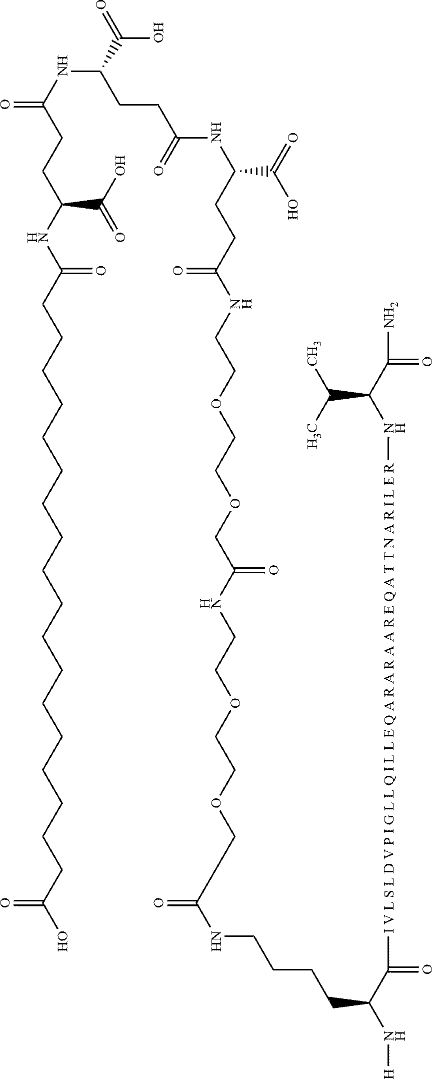

SEQ ID NO: 7

LCMS:
Calculated mass: M/3 = 1780.37; M/4 = 1335.52; M/5 = 1068.62
Found mass: M/3 = 1780.43; M/4 = 1335.57; M/5 = 1068.66

### Compound 8 - Reference Compound

N{ε}-[[[(4S)-4-carboxy-4-[[(4S)-4-carboxy-4-[[(4S)-4-carboxy-4-(19-carboxynonadecanoylamino)butanoyl]amino]butanoyl]amino]butanoyl]amino]ethoxy]ethoxy]a cetyl]amino]ethoxy]ethoxy]acetyl]Lys-[Glu37]-hUcn2

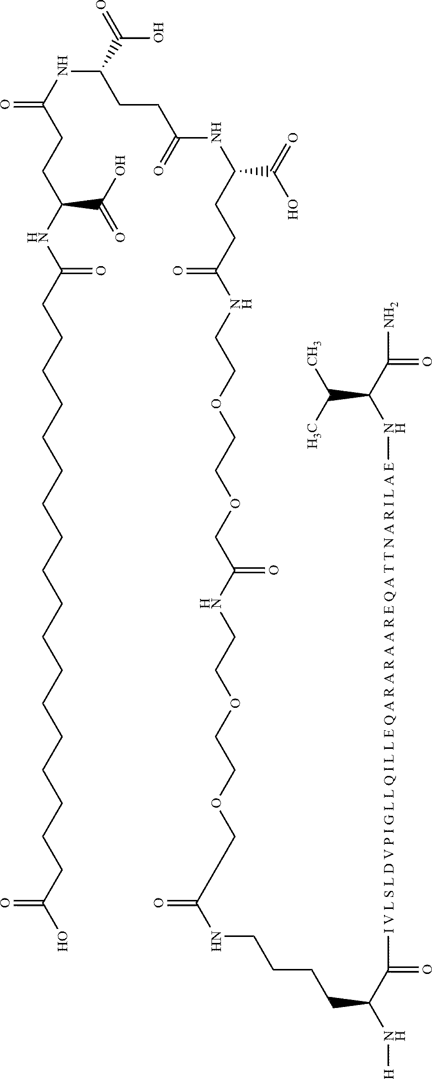

SEQ ID NO: 8

LCMS:
Calculated mass: M/4 = 1314.26; M/5 = 1051.61
Found mass: M/4 = 1314.27; M/5 = 1051.61

### Compound 9 - Reference Compound

N{ε}-[[[(4S)-4-carboxy-4-[[(4S)-4-carboxy-4-[[(4S)-4-carboxy-4-(19-carboxynonadecanoylamino)butanoyl]amino]butanoyl]amino]butanoyl]amino]ethoxy]ethoxy]a cetyl]amino]ethoxy]ethoxy]acetyl]Lys-[Glu35]-hUcn2

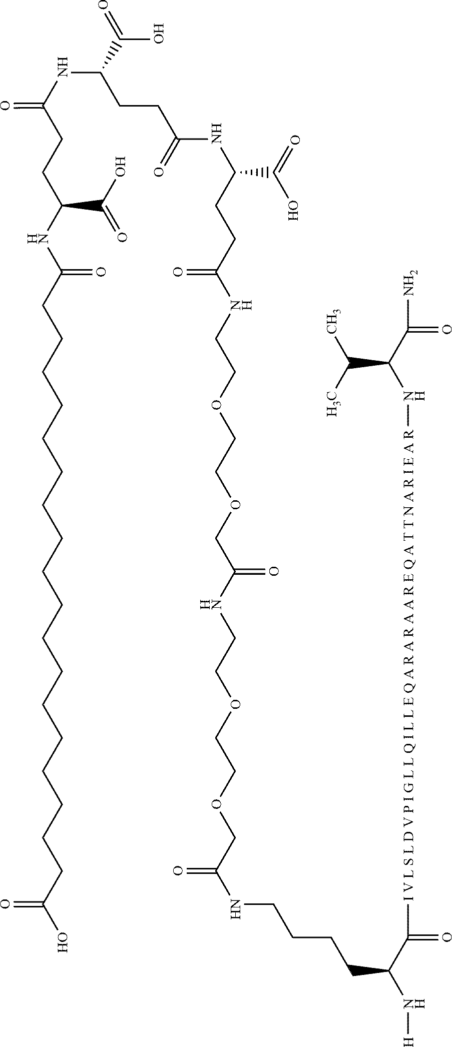

SEQ ID NO: 9

LCMS:
Calculated mass: M/3 = 1766.35; M/4 = 1325.01; M/5 = 1060.21
Found mass: M/3 = 1766.40; M/4 = 1325.05; M/5 = 1060.23

### Compound 10 - Reference Compound

N{ε}-[[[(4S)-4-carboxy-4-[[(4S)-4-carboxy-4-[[(4S)-4-carboxy-4-(19-carboxynonadecanoylamino)butanoyl]amino]butanoyl]amino]butanoyl]amino]ethoxy]ethoxy]a cetyl]amino]ethoxy]ethoxy]acetyl]Lys-[Glu33,Glu35]-hUcn2

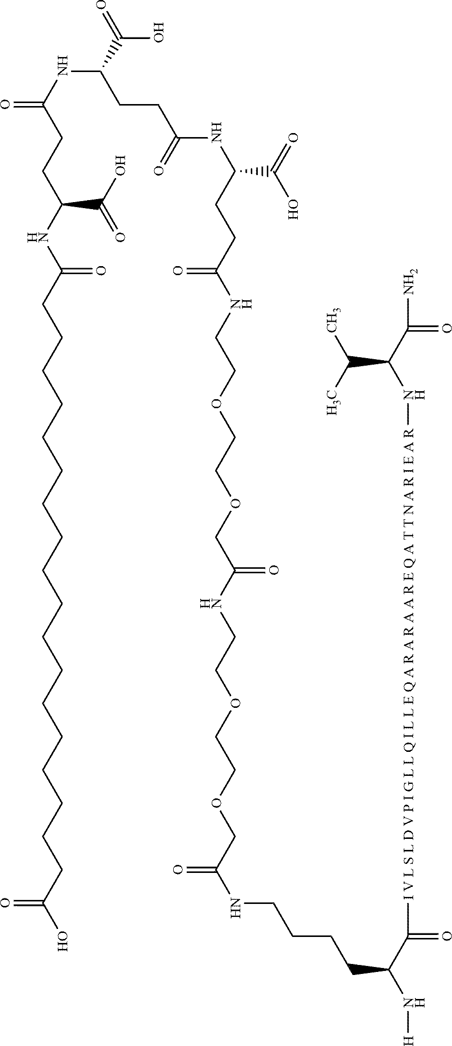

SEQ ID NO: 10

LCMS:
Calculated mass: M/3 = 1757.33; M/4 = 1318.29; M/5 = 1054.80
Found mass: M/3 = 1757.38; M/4 = 1318.29; M/5 = 1054.83

### Compound 11 - Reference Compound

N{ε1}-[[[(4S)-4-carboxy-4-[[(4S)-4-carboxy-4-[[(4S)-4-carboxy-4-(19-carboxynonadecanoylamino)butanoyl]amino]butanoyl]amino]butanoyl]amino]ethoxy]ethoxy]a cetyl]amino]ethoxy]ethoxy]acetyl]-[Lys1,Glu33,Glu36]-hUcn2

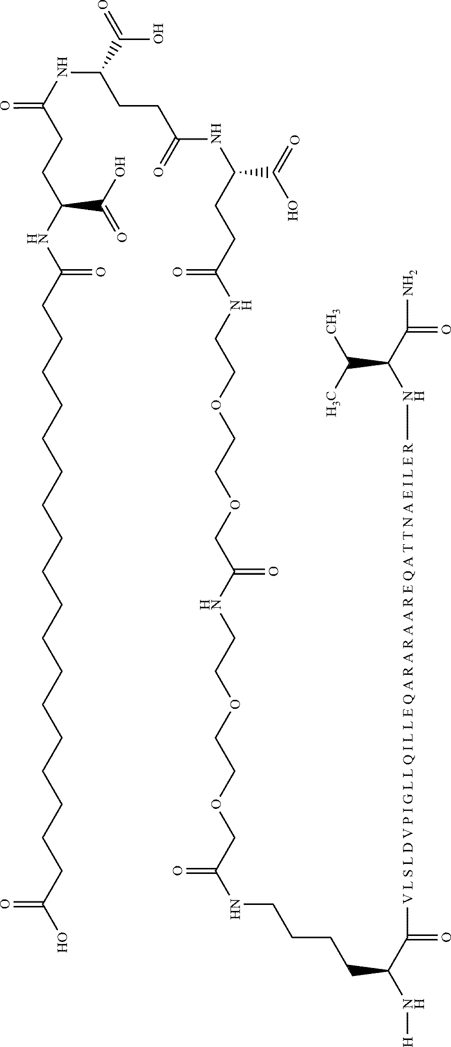

SEQ ID NO: 11

LCMS:
Calculated mass: M/3 = 1733.65; M/4 = 1300.49; M/5 = 1040.59
Found mass: M/3 = 1733.71; M/4 = 1300.54; M/5 = 1040.63

### Example 2: CRF₁ and CRF₂ Receptor Binding

The purpose of this example was to test the in vitro binding of the compounds of the invention to receptor subtypes CRF₁ and CRF₂. The in vitro binding of the compounds was determined in a scintillation proximity assay (SPA) as described below. The native, human Ucn1 and Ucn2 peptides are included as references.

Scintillation Proximity Assay: CHO-K1 cells expressing either the CRF₁ receptor or the CRF₂ receptor were obtained from DiscoveRx. Both cell lines were cultured at 37° C. in a humidified atmosphere with 5% CO₂. The culture media used was Ham’s F-12 Nutrient Mix with Glutamax (Gibco) containing 10% fetal bovine serum, 1% Pen/Strep, 800 µg/ml G418 and 300 µg/ml Hygromycin B. Membranes were prepared by the following protocol: cultured cells were washed twice with cold Dulbecco’s phosphate buffered saline and detached using TrypLE™ (ThermoFisher) and transferred to tubes and centrifuged for 5 minutes at 1000 g at +4° C. Pellets were resuspended in ice cold homogenization buffer (20 mM Hepes pH 7.1, 5 mM MgCl₂, 1 mg/ml Bacitracin with 2 complete EDTA-free protease inhibitor cocktail tablets/50 ml (Roche)) and then homogenized for 30 seconds using a Ultra-Turrax T25 tissue homogenizer (IKA) at medium speed. The homogenate was centrifuged at 35,000 g for 15 minutes at +4° C. using an ultracentrifuge, and the supernatant was discarded, and fresh homogenization buffer was added. Homogenization of the pellet was repeated a total of three times. The final pellet was resuspended in a few millilitres of homogenization buffer and protein concentration was determined using the Bradford method. The membrane preparation was transferred to cryotubes, and the aliquots were stored at -80° C. Each membrane preparation was subsequently titrated to give an appropriate window in the assay.

Test procedure: Receptor SPA binding assays were performed in white 96-well plates in a total volume of 200 µl per well. Freeze dried analogues were dissolved in 80% dimethyl sulfoxide, 20% H₂O and serial dilutions (1:10) were performed in binding buffer (20 mM Tris-HCl pH 7.4, 5 mM MgAc₂, 2 mM EGTA and 0.1% ovalbumin); the final assay concentrations ranging from 1 µM to 1 pM. Analogues and 7.5-50 µg of CRF₁ receptor membranes/well or 0.5 µg of CRF₂ receptor membranes/well were added to assay plates. A mix of wheat germ agglutinin coated SPA beads (PerkinElmer) and [¹²⁵I]-Sauvagine (PerkinElmer), both in binding buffer, was added to yield 0.4 mg beads/well and 50,000 cpm of radio ligand per well. Plates were sealed and incubated at 25° C. for 2 hours in a plate shaker set at 400 rpm and thereafter centrifuged at 1500 rpm for 10 minutes. SPA plates were let to stand at room temperature for about 16 hours prior to reading on microplate scintillation counter. Displacement of radioligand was measured as reduction in luminescence, and IC₅₀ values were calculated by nonlinear regression analysis of four parameter sigmoidal dose-response curves.

TABLE 1

Receptor binding affinity

| Compound No. | Human CRF₁ receptor IC50 [nM] | Human CRF₂ receptor IC50 [nM] |
|---|---|---|
| Compound 1 - hUcn1 | 0.07 | 0.12 |
| Compound 2 - hUcn2 | 447 | 0.58 |
| Compound 3 | >1000 | 1.14 |
| Compound 4 | >1000 | 1.47 |
| Compound 9 - reference compound | >1000 | 897 |
| Compound 10 - reference compound | >1000 | 569 |
| Compound 11 - reference compound | >1000 | 196 |

In contrast to the reference compounds, the compounds of the invention were associated with high binding affinity to the human CRF₂ receptor and low binding affinity to the human CRF₁ receptor. The results demonstrated that E substitution in position 33 together with E substitution in either 36 or position 37 was associated with surprisingly high CRF receptor affinity and selectivity.

### Example 3: In Vitro Functional Potency (β-Arrestin)

The purpose of this investigation was to test the in vitro agonistic activity (potency) of the compounds of the invention to receptor subtype CRF₁ and CRF₂. The in vitro potency of the compounds was determined in a human CRF₁ receptor and human CRF₂ receptor CHO Cell Arrestin Recruitment Assay as described below. The native, human Ucn1 and Ucn2 peptides were included as references.

Assay principle: Activated G-protein coupled receptors (GPCRs) can interact with the Arrestin family of signalling proteins. The potency of peptides for CRF induced Arrestin recruitment is determined using the PathHunter Enzyme Fragment Complementation approach substantially as described in von Degenfeld et al., FASEB J., 2007, 3819, 26 and Hamdouchi et al., J. Med. Chem., 2016, 59, 10891-10916. The target GPCR is tagged with the small fragment of β-gal called ProLink™, a low affinity version of enzyme donor and co-expressed in cells stably expressing β-Arrestin tagged with enzyme acceptor. Activation of the GPCR stimulates binding of β-Arrestin to the ProLink-tagged GPCR, forcing complementation of ProLink and enzyme acceptor, resulting in the formation of an active β-gal enzyme. The resulting active enzyme hydrolyses substrate present in the PathHunter detection reagents to generate light.

Cell cultivation: CHO-KI cells expressing Pro-Link-tagged human CRF₁ or CRF₂ receptors and enzyme-acceptor-tagged arrestin-2 was obtained from DiscoveRx - catalogue numbers 93-225C2 (CRF₁) and 93-0251C2 (CRF₂). The assay media was Opti-MEM (Gibco) containing 0.1% Ovalbumin (Sigma). The cells were grown according to the protocol outlined below using the AssayComplete Cell Culture Kit-107 (DiscoveRx 92-3107G). The following procedures were followed:
1) Individual components of AssayComplete Cell Culture Kit were thawed in a 37° C. water bath.
2) Each component separately we mixed by gently inverting bottles prior to use under the tissue culture hood.
3) Using aseptic techniques, complete medium were prepared by adding the entire contents of Component A and Component B to the 500 mL of AssayComplete Cell Culture Reagent.
4) Appropriate selection antibiotic(s) were added to the AssayComplete Cell Culture Reagent - Typically use 0.3 mg/ml Hygromycin B (Discoverx 92-0029) and 0.8 mg/ml G418 (Discoverx 92-0030)

Cells were cultured at 37° C. in a humidified atmosphere with 5% CO₂ in standard Tissue Culture Flasks (VWR 10861-572) according to the protocol below:
1) Once cells are confluent, spent media was removed by aspiration, then washed by adding ~25 ml of phosphate buffered saline to remove residual serum and then the phosphate buffered saline was removed by aspiration.
2) The cells were detached by adding enough Cell Detachment reagent (DiscoverX 92-0009) to cover bottom of flask, flasks were kept at 37° C., checked every 2 minutes until cells were detached.
3) Cell plating reagent (Discoverx 93-0563R2A) is added, ~25 ml, cell suspension was pipetted into 50 ml falcon tubes.
4) Cells were centrifuged for 3 minutes at 1200 rpm.
5) Cells were counted using a NucleoCounter (NC-200, Cemometec)
6) Cells were resuspended to 0.1 million cells per ml in Cell plating reagent (Discoverx 93-0563R2A) for plating into 384 well plates.

Test procedure: The cells were plated into 384 white PDL coated microplates (Greiner Bio One 781945) the day before running the assay. The cells were plated at 5000 cells per well in 50 µL of cell plating reagent. The day of the assay, the spent media was removed using the Blue Washer (1573-L BlueCatBio). The media was replaced with 10 µL of Optimem (1105821 Gibco) with 0.1% ovalbumin (Sigma). Peptides were solubilized in 80% DMSO, 20% water and serial dilutions were performed using the Echo acoustic dispenser (Echo 550 Beckman-Coulter). The peptides were added directly to the cell plates using the Echo acoustic dispenser. Plates were first incubated for 90 minutes in a 37° C., 5% CO₂ incubator and then 5 µL of PathHunter detection reagent was added (DiscoveRx, 93-0001) and the plates were incubated at room temperature for 60 minutes after which the luminescence signal was measured (BMG Pheristar). Peptide concentration-response curves were fitted to a four-parameter logistic model to calculate potency as an EC50.

TABLE 2

Receptor potency

| Compound No. | Human CRF₁ receptor EC50 [nM] | Human CRF₂ receptor EC50 [nM] |
|---|---|---|
| Compound 1 - hUcn1 | 22 | 31 |
| Compound 2 - hUcn2 | >10000 | 14 |
| Compound 3 | >10000 | 55 |
| Compound 4 | >10000 | 42 |

As shown in Table 2, the compounds of the invention demonstrated potent, functional activation of the human CRF₂ receptor while no detectable activation was observed for the human CRF₁ receptor corroborating that the compounds of the invention are potent and selective CRF₂ receptor activators.

### Example 4: Pharmacokinetic Studies in Minipigs After Intravenous Administration

The purpose of this investigation was to determine the in vivo half-life of the compounds of the invention after i.v. administration to minipigs using a pharmacokinetic (PK) study in Göttingen Minipigs assessing terminal half-life. The expression ‘terminal half-life’ as used herein, and means the time period required to reduce the plasma concentration by 50%, measured after the initial distribution phase.

Animals and housing: Female Göttingen Minipigs, weighing approximately 15-25 kg, were obtained from Ellegaard Minipigs, Dalmose Denmark. The minipigs were housed individually in the Animal Unit at BioAdvice A/S, ∅lstykke Denmark (later Minerva Imaging A/S) and were fed restrictedly twice daily with Altromin 9033 (Chr. Petersen A/S, Ringsted, Denmark). After minimum 2 weeks of acclimatization two permanent central venous catheters were implanted during general anaesthesia in either the caudal or cranial caval vein in each animal. The animals were allowed approximately 1 week of recovery after the surgery and were then used for repeated pharmacokinetic studies with a suitable wash-out period between successive dosing episodes.

Body Weight: The animals were weighed weekly and in addition, either on the dosing day or the day before dosing in order to decide the correct dosing volume. The minipigs typically weighed between 20-30 kg on the dosing days.

Sample: Test compound were prepared in the following buffer: 8 mM phosphate, 250 mM glycerol, 0.007% polysorbate 20, pH 7.4.

Administration of peptides and dosing solutions: There were no food restrictions during the study and the animals had ad libitum access to water during the whole study period. Intravenous injection of the test compound was given through one of the central-venous catheter, which was flushed with minimum 10 mL of sterile saline post administration. The test substances were dosed in a dose of 2 nmol/kg and with a dose volume of 0.1 mL/kg (n=3). Multiple test compounds were dosed together (cassette-dosing, with separate formulations dosed consecutively) in each pig. Blood samples of 1.3 mL were taken through the central-venous catheter (typically the one that had not been used for dosing) at the following time points in relation to the dosing: Predose, 0.083, 0.25, 0.5, 1, 1.5, 2, 3, 4, 6, 8, 10, 24, 30, 48, 72, 96, 144, 168, 192, 216, 240, 264 and 312 h. After each blood sample the catheter was flushed with minimum 5 ml of sterile 0.9 % NaCl containing 10 IE/mL heparin. Aseptic technique was demanded to avoid bacterial growth in the catheter that otherwise increases the risk of clot formation in the catheter. Immediately after sampling, the blood was transferred to test tubes containing K₃EDTA buffer (8 mM). The tubes were kept on wet ice until centrifugation for 10 min at 4° C. and approx. 2000 × g within 0.5 h after sampling. Afterwards, plasma (min. 200 µL) was transferred to Micronic tubes on dry ice and kept at -20° C. until analysis. The plasma samples were analysed as described below.

Quantitative Assay for Plasma Samples: The test compound was assayed in plasma by turbulent flow chromatography coupled to liquid chromatography mass spectrometric (LCMS) detection. The selectivity of the method allowed various test compounds to be quantitated in one sample, e.g. cassette dosing. Calibrators were prepared by spiking blank plasma with test compounds covering a range from 0.5 to 2000 nM. One volume of calibrator, blank plasma or study samples was diluted with three volumes of 96% ethanol in order to precipitate the majority of plasma proteins. The ethanol contained about 25 nM of an internal standard (IS). Samples were mixed for 3 min at room temperature and centrifuged for 30 min at 4600 rpm at 4° C. Supernatants were transferred into new tubes and diluted with 2 volumes of 1 % formic acid. The diluted samples were analysed by turbulent flow chromatography LCMS using a Cyclone turboflow column (0.5 × 50 mm, ThermoFisher Scientific) for sample clean-up and one of the following analytical columns: Aeris Peptide XB-C18 100 Å (50 × 2.1 mm, 3.6 µm, Phenomenex), XBrigde C8 300 Å (2.1 × 50 mm, 3.5 µm, Waters) or XBrigde Peptide BEH C4 300 Å (2.1 × 50 mm, 3.5 µm, Waters). A gradient elution was applied using mobile phase A (consisting of milli-Q water with 1% vol/vol formic acid and 5% vol/vol methanol/acetonitrile (50/ 50, vol/vol) and mobile phase B (consisting of 95% vol/vol methanol/acetonitrile (50/ 50, vol/vol) with 1% vol/vol formic acid and 5% vol/vol milli-Q water). Typical gradient conditions were from 65% to 85% mobile phase B. MS-measurements were performed on a Q-Exactive mass spectrometer (ThermoFisher Scientific) in single ion monitoring mode for the +4 or +5 charge state of each test compound and for the internal standard. Analysis of MS raw data was carried out with the QuanBrowser software (Thermo Fisher Scientific). Intensities of the first 4-5 isotopic peaks of the measured test compounds were extracted and integrated over the elution profile of the peak. From the peak areas of the calibrator samples, calibration curves were calculated by linear regression analysis and used to determine the plasma concentration of the test compounds in the study samples. The integrated ion intensities of the IS peak in each sample were used for normalisation. Quality control samples were included between and after the study samples. The deviation between nominal and calculated concentration of the calibrators and quality control samples was below 20%.

Data management: The individual plasma concentration-time profiles were analysed by a non-compartmental pharmacokinetics analysis using Phoenix WinNonlin (Pharsight Inc., Mountain View, CA, USA). Terminal half-life is given as the harmonic mean in Table 3.

TABLE 3

Terminal half-life

| Compound No. | Terminal half-life [h] |
|---|---|
| Compound 3 | 96 |
| Compound 4 | 103 |

As shown in Table 3, the compounds of the invention have very long half-lives as compared to the half-life of hUcn2, which e.g. has been measured to have a half-life of approximately 10 min in humans (Davies et al. J. AM. Coll. Cardiol. 2007, 49(4), 461-471). The measured half-lives in minipigs predicts half-lives in humans sufficient for at least once-weekly administration via liquid injection, based on knowledge from other acylated peptides (see e.g. Lau et al. J. Med. Chem. 2015, 58, 7370-7380 and Hijazi Eur. J. Drug Metabolism and Pharmacokinetics 2021, 46, 163-172).

### Example 5: Acute Food Intake Reduction in Ad Libitum Fed Rats

The purpose of this study was to determine the effect of a single subcutaneous dose of the compounds of the invention on food intake in rats over at least two days after dosing.

Animals and housing: Sprague Dawley (SD) rats from Taconic Europe, Denmark were used for the acute food intake experiments. The rats were housed in the Animal Facility at Novo Nordisk A/S and had ad libitum access to food (Research diet, LF 10% (D12450B)) and water throughout both the acclimatisation and the study period. The rats arrived at least 9 days before the start of the experiment to allow acclimatisation to the experimental settings. Approx. 2 days after arrival, the rats were put on a reversed light cycle (dark from 11 am -11 pm) and placed in the HM2 system. In the HM2 system the rats are identified by a chip, which allow individual food intake measurements in group-housed rats. Typically, three rats were housed in each cage in the HM-2 system, with the 3 rats being assigned to 3 different groups in order to avoid cage effects and to avoid loss of too many data if one cage should malfunction.

Body weight: The rats were weighed on the day of dosing and at the end of the experiment (48 h after dosing) and had an average body weight of approx. 300 g at the start of the experiment (day of dosing).

Samples: The test compound was formulated in a concentration of 100 nmol/kg in the following buffer: 8 mM phosphate, 250 mM glycerol, 0.007% polysorbate 20, pH 7.4.

Dosing: Rats were dosed in the morning right before lights were turned off. Each test compound was tested in a dose of 100 nmol/kg and the group size was 9. In addition, a vehicle group of 10 rats was included. The rats were dosed once according to body weight with a dose volume of 1 mL/kg. The time of dosing was recorded for each group. After dosing, the rats were returned to their home cages, where they had ad libitum access to food and water. The food consumption was recorded individually and continuously by on-line registration (HM2 system or BioDaq) for up to 48-72 h. At the end of the experimental session, the animals were euthanised.

Data management: Food intake was measured in periods of 24 h from 0-24 and 24-48 h after dosing and the percent reduction in each dose group compared to average vehicle food intake in the same two periods was calculated. Table 4 shows the food intake reduction compared to vehicle on day 1 and day 2 after dosing.

TABLE 4

Acute food intake reduction relative to vehicle

| Compound No. | Day 1 (0-24 h) [%] | Day 2 (24-48 h) [%] |
|---|---|---|
| Compound 3 | -35 | -38 |
| Compound 4 | -50 | -43 |

As shown in Table 4, treatment with the compounds of the invention lead to reduction in food intake as compared with vehicle treatment.

Example 6: Pharmacodynamic study in diet-induced obese (DIO) rats The purpose of this study was to evaluate the effect of the test compound on food intake, body weight, body composition, oral glucose tolerance, muscle mass and biomarkers after sub-chronic dosing for approx. 3 weeks to DIO rats.

Animals and housing: Male DIO Sprague Dawley rats from Charles River, France were housed in the Animal Facility at Novo Nordisk A/S and had ad libitum access to food (D12451 Research Diet 45% fat) and water throughout both the acclimatisation and the study period. The rats arrived at least 1 month before the start of the experiment to allow acclimatisation. The rats were single-housed in a reverse light cycle (dark from 11 am -11 pm) for at least 2 weeks prior to experimental start, and were handled regularly until study start in order to socialize the rats and reduce stress during the experiment.

Groups: Two studies were run in DIO rats. In Study 1, the rats were allocated into the following treatment groups based on body weight and body fat: Group 1: Vehicle (n=8); Group 2: Compound 4, 30 nmol/kg (n=8); Group 3: Compound 4, 100 nmol/kg; Group 4: Compound 4, 300 nmol/kg (n=8). The average body weight of the rats was 769 g on Day 1 of the study. In Study 2, the rats were allocated into two groups: Group 1: Vehicle (n=8); Group 2: Compound 4, 300 nmol/kg (n=8). The average starting body weight (on Day 1) was 679 g.

Formulation: The test compound was formulated in the following buffer: 8 mM phosphate, 8 mM phosphate, 270 mM D-sorbitol, 0.007 % polysorbate 20, pH 7.4. The concentration of test compound was 27.6, 91.7 and 283.6 nmol/mL, for Group 2, 3 and 4, respectively.

Dosing: The rats were weighed and dosed s.c. once daily at 10.30 am (Study 2) or 11.30 am (Study 1) according to this BW. In Study 1, the rats were treated with approx. ⅓ of the final dose level of test compound on Day 1, whereafter the full dose was given on the remaining days. In Study 2, the rats received the full dose on all dosing days.

Analysis: Body weight and food intake was measured manually once daily; the latter by weighing the food given and the food left. Before study start (day -2) and at study end on Day 19 (Study 1) or Day 23 (Study 2) the body composition was determined using QMR scanner (EchoMRI from Echo Medical Systems Huston, Texas (EchoMRl-036)). In study 2, the rats were subjected to a combined gastric emptying and oral glucose tolerance test (OGTT), which was performed after a 4 h fast (food removed at 6 am). At time zero, 2 g glucose/kg was given p.o. with a sonde together with 100 mg paracetamol/kg (corresponding to 4 mL/kg of a 500 g/L glucose solution with 50 mg/mL paracetamol). Blood samples of 10 µL were collected from the tail vein by venipuncture in capillary tubes coated with sodium-heparin at the following time points in relation to dosing of the glucose bolus: T=0, 15, 30, 60, 90, 120 and 180 min. The blood was immediately mixed with 500 µL of Biosen buffer and kept on wet ice until analysis for blood glucose content on a Biosen S-line analyzer (EKF-diagnostic GmbH, Barleben, Germany) according to the manufacturer’s instructions and within 6 h from sampling. In addition, larger blood samples of 250 µL were taken from the sublingual vein in EDTA tubes at the following time points during the OGTT: predose, 30, 60 and 120 min. The blood was kept on wet ice until centrifugation for 5 min at 6000 rpm and 4° C. within 0.5 h from sampling. Hereafter the plasma was aliquoted, frozen on dry ice and kept at -20° C. until analysis for plasma insulin and other biomarkers. The rat insulin content was measured using homogenous Luminescence Oxygen Channeling Immunoassay assay. During the assay, a concentration dependent bead-analyte-immune complex was created, resulting in light output which was measured on a Perkin Elmer Envision reader. In the assay, (anti rat insulin) mAb D3E7-conjugated acceptor-beads and biotinylated mAb D6C4 (also raised against rat insulin) were used together with generic streptavidin-coated donor beads. The lower limit of quantification of the assay was 35 pM. The animals in Study 1 were euthanised by bleeding under isoflurane anaesthesia on Day 23 and the extensor digitorum longus muscle was dissected and weighed. The animals in Study 2 were euthanised on Day 25 using CO₂/O₂.

Data management: Data was managed in GraphPad Prism v. 9.0.1 for Windows (GraphPad Software, San Diego, California USA). Average daily food intake from day 1 to day 22, baseline corrected body weight on day 22, EDL muscle weight, baseline-corrected fat mass and lean mass were calculated, as well as the area under the curve for the blood glucose and insulin during the OGTT in Study 2. Results are reported in Table 5.

TABLE 5

Central body measures and metabolic parameters in DIO rats dosed once daily with Compound 4 for approx. 3 weeks

| Parameter | Study | Vehicle | 30 nmol/kg | 100 nmol/kg | 300 nmol/kg |
|---|---|---|---|---|---|
| BW change from baseline (%) (day 22) | 1 | -0.4 | -0.2 | -2.3 | -0.3 |
| Fat mass change from baseline (g) | 1 | 14.5 | -35.5 | -42.6 | -45.9 |
| Lean mass change from baseline (g) | 1 | 3.3 | 29.7 | 12.8 | 30.3 |
| Weight of the EDL muscle (g) | 1 | 0.27 | 0.36 | 0.38 | 0.35 |
| Average daily food intake (g) | 1 | 21 | 17.7 | 15.9 | 16.7 |
| Fasting plasma insulin (pM) | 2 | 552 | ND | ND | 299 |
| Fasting blood glucose (mM) | 2 | 5.3 | ND | ND | 5.0 |
| AUC_{glucose}(₀₋₁₈₀ₘᵢₙ₎ (mM*min) | 2 | 1119 | ND | ND | 1053 |
| AUCᵢₙₛᵤₗᵢₙ₍₀₋₁₈₀ₘᵢₙ₎ (pM*min) | 2 | 94988 | ND | ND | 62685 |

*BW: body weight
EDL=extensor digitorum longus
ND: Not determined

As shown in Table 5, treatment with Compound 4 lead to a decrease in average daily food intake and fat mass and an increase in lean mass. There were no differences between the groups in the body weight change from Day 1 to Day 22. Fasting insulin and AUCᵢₙₛᵤₗᵢₙ during the OGTT were both lower in the animals treated with Compound 4 compared to vehicle, whereas there was only a small decrease in fasting blood glucose and in AUC_{glucose} during the OGTT. Altogether, treatment with Compound 4 induced a significant improvement in body composition and in parameters related to glucose metabolism, supporting the potential as a treatment for obesity and type 2 diabetes.

### Example 7: Physical Stability

The purpose of the study was to evaluate the physical stability of a liquid pharmaceutical formulation comprising test compound. The physical stability was determined as the formation of visible particles and the change in turbidity over time.

Test procedures: Test compound was dissolved in 8 mM sodium phosphate, pH 7.4 to a concentration of 31-37 mg/mL in vials (except for Compound 7 which was dissolved to a concentration of 21 mg/mL and Compound 5 which was not soluble at concentrations of ≥20 mg/mL). The vials were incubated at 37° C. for 2 weeks. Every day the vials were rotated end-to-end for 1 minute, 30 rounds. Every second day the vials were placed in front of a black background with strong LED light (Schott light source, KL2500) illuminating from below and a picture for analysis was taken.

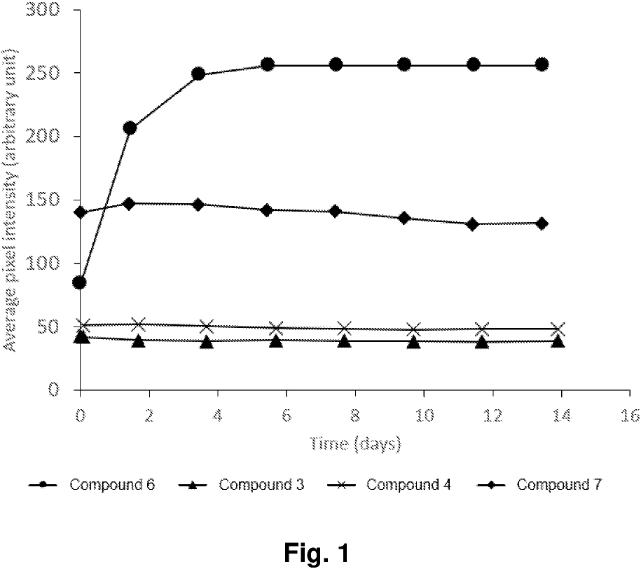

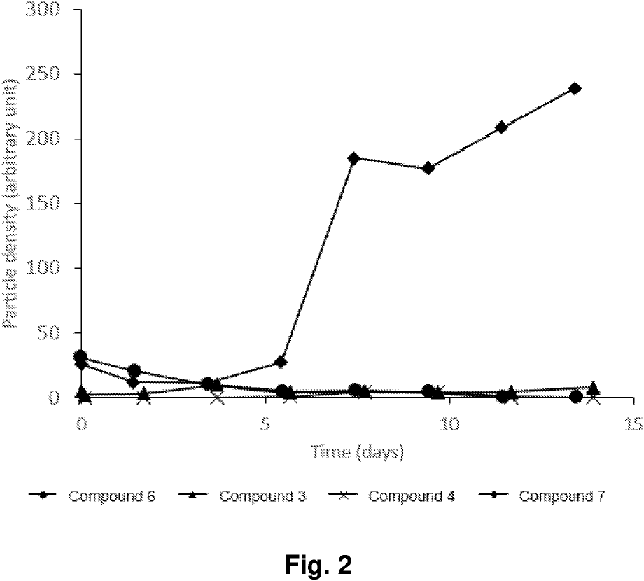

Analysis: “Average pixel intensity” is a measure of turbidity of a liquid solution. It was measured by taking the average pixel intensity, ranging from 0 (black) to 255 (white), in a small square of a digital image of the glass vials containing a liquid solution and results are reported in FIG. 1. “Particle density” is a measure of visible particles in a liquid solution. It was measured by counting the number of visible particles in a region of a digital image of the glass vial containing a liquid solution and dividing with the area of the region and the results are reported in FIG. 2. “Particle formation onset” describes the first day on which visual particles were observed and results are reported in Table 6.

TABLE 6

Particle formation onset

| Compound No. | Particle formation onset [day] |
|---|---|
| Compound 3 | No particles observed |
| Compound 4 | No particles observed |
| Compound 5 - reference compound | Not soluble |
| Compound 6 - reference compound | 1 |
| Compound 7 - reference compound | 7 |

The pharmaceutical formulations comprising the compounds of the invention were fully soluble at test conditions, they were not associated with increased turbidity (FIG. 1), and they were not associated with formation of visible particles (FIG. 2 and Table 6). The results demonstrated that the introduction of E substitution resulted in a high solubility, and that E substitution in position 33 together with E substitution in either position 36 or position 37 resulted in a high physical stability.

### Example 8: Chemical Stability

The purpose of the study was to evaluate the chemical stability of a liquid pharmaceutical formulation comprising test compound. The chemical stability was be determined by measuring test compound purity loss (i.e. reduction of test compound relative to other species in the sample) over time using ultra-performance liquid chromatography (UPLC) coupled to an ultra-violet detector operated in reversed-phase mode.

Equipment and analysis conditions: Acquity UPLC I-Class instrument (Waters, Milford, US) together with either an Acquity UPLC CSH Fluorophenyl column (PN. 186005353 from Waters, Milford, US) or an Acquity Premier CSH column (PN. 186009462 from Waters, Milford, US), both having the dimensions 150 × 2.1 mm, particle diameter 1.7 µm and kept at 30° C., was used for the test. Mobile phase A was 0.1% vol/vol trifluoroacetic acid in water and mobile phase B was 0.09% vol/vol trifluoroacetic acid in acetonitrile. The flow rate was 0.35 mL/min while elution was done with a linear gradient of 26% to 40% mobile phase B over 50 min for the UPLC Fluorophenyl column and 30% to 50% mobile phase B over 50 min for the Premier CSH column. Detection was performed at UV 214 nm with 2 µL of volume injected from each sample.

Test procedures: Test compound was formulated in an 8 mM sodium phosphate buffer at pH 7.4 at a concentration of 0.7-1.1 mM (except for Compound 5 which was not soluble at concentrations of ≥0.7 mM). Samples were kept in glass vials for quiescent storage in a 37° C. incubation chamber and subjected to analysis after 0, 1, 2, 3 and 4 weeks. Samples were analysed using UPLC (as described above) with chromatogram peak area as a measure of test compound concentration. Purity was determined at each timepoint as the fraction, given in %, of test compound relative to other species present in the sample. The purity loss over time is the relative difference in % test compound between timepoints and it is reported in Table 7 as the average from the five timepoints.

TABLE 7

Chemical stability

| Compound No. | Average purity loss per week [%] |
|---|---|
| Compound 2 - hUcn2 | 6.15 |
| Compound 3 | 1.21 |
| Compound 4 | 0.91 |
| Compound 5 - reference compound | Not soluble |
| Compound 6 - reference compound | 0.90 |
| Compound 7 - reference compound | 3.95 |
| Compound 8 - reference compound | 2.43 |

The pharmaceutical formulations comprising the compounds of the invention were fully soluble at test conditions and they were associated with low purity loss (Table 7). The results demonstrated that the introduction of E substitution resulted in a high solubility, and that introduction of E substitution in position 33 (e.g. together with introduction of E substitution in either position 36 or position 37) resulted in a high chemical stability.

While certain features of the invention have been illustrated and described herein, many modifications, substitutions, changes, and equivalents will now occur to those of ordinary skill in the art. It is, therefore, to be understood that the appended claims are intended to cover all such modifications and changes as fall within the true spirit of the invention.

1. A compound comprising a peptide of the sequence:
K-I-V-L-S-L-D-V-P-I-G-L-L-Q-I-L-L-E-Q-A-R-A-R-A-A-R-E-Q-A-T-T-N-A-E-I-L-X₃₆-X₃₇-V;
wherein
X₃₆ is A or E,
X₃₇ is R or E;
wherein at least one of X₃₆ and X₃₇ is E;
wherein a substituent is attached to the N-terminal K of the peptide;
or a pharmaceutically acceptable salt thereof.

2. The compound according to claim 1, with the proviso that only one of X₃₆ and X₃₇ is E.

3. The compound according to claim 1, wherein the C-terminus of the peptide optionally is amidated.

4. The compound according to claim 1, wherein the substituent is attached via an amide bond to the epsilon amino group of the N-terminal K.

5. The compound according to claim 1, wherein the substituent comprises Chem. 1: 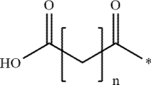
, wherein n is an integer in the range of 8-20.

6. The compound according to claim 5, wherein n is an integer in the range 16-18.

7. The compound according to claim 5, wherein n is 18.

8. The compound according to claim 5, wherein the substituent further comprises three residues of Chem. 2 and two residues of Chem. 3, 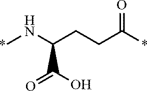 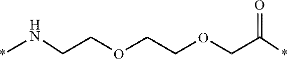
.

9. The compound according to claim 1, wherein the substituent is 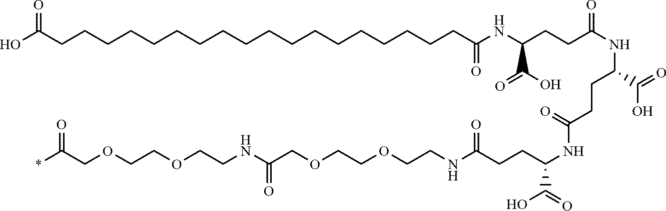 [SMILES: *C(=O)COCCOCCNC(=O)COCCOCCNC(=O)CC[C@H](NC(=O)CC[C@H](NC(=O)CC[C@H](NC(=O)CCCCCCCCCCCCCCCCCCC(=O)O)C(=O)O)C(=O)O)C(=O)O]
.

10. The compound according to claim 1, wherein the peptide is a Ucn2 analogue.

11. The compound according to claim 1, wherein the compound is a Ucn2 derivative.

12. The compound according to claim 1, wherein the compound is selected from the group consisting of Compound 3 and Compound 4; or an pharmaceutical acceptable salt thereof.

13. A method of treating type 2 diabetes, comprising administering the compound according to claim 1 to a subject in need thereof.


SEQUENCE LISTING (11 sequences)

SEQ ID NO: 1 - protein, 40 aa, Homo sapiens
DNPSLSIDLTFHLLRTLLELARTQSQRERAEQNRIIFDSV

SEQ ID NO: 2 - protein, 38 aa, Homo sapiens
IVLSLDVPIGLLQILLEQARARAAREQATTNARILARV

SEQ ID NO: 3 - protein, 39 aa, synthetic construct
KIVLSLDVPIGLLQILLEQARARAAREQATTNAEILAEV

SEQ ID NO: 4 - protein, 39 aa, synthetic construct
KIVLSLDVPIGLLQILLEQARARAAREQATTNAEILERV

SEQ ID NO: 5 - protein, 39 aa, synthetic construct
KIVLSLDVPIGLLQILLEQARARAAREQATTNARILARV

SEQ ID NO: 6 - protein, 39 aa, synthetic construct
KIVLSLDVPIGLLQILLEQARARAAREQATTNAEILARV

SEQ ID NO: 7 - protein, 39 aa, synthetic construct
KIVLSLDVPIGLLQILLEQARARAAREQATTNARILERV

SEQ ID NO: 8 - protein, 39 aa, synthetic construct
KIVLSLDVPIGLLQILLEQARARAAREQATTNARILAEV

SEQ ID NO: 9 - protein, 39 aa, synthetic construct
KIVLSLDVPIGLLQILLEQARARAAREQATTNARIEARV

SEQ ID NO: 10 - protein, 39 aa, synthetic construct
KIVLSLDVPIGLLQILLEQARARAAREQATTNAEIEARV

SEQ ID NO: 11 - protein, 38 aa, synthetic construct
KVLSLDVPIGLLQILLEQARARAAREQATTNAEILERV